SEQUENCE LISTING (70 sequences)

SEQ ID NO: 1 - DNA, 62 bp, Artificial Sequence
  Chemically synthesized
tgggcactatttatatcaacnnnnnnnnnnnnnnnnnnnnnnnnnaatgtcgttggtggccc

SEQ ID NO: 2 - DNA, 35 bp, Artificial Sequence
  Chemically synthesized
cccgacacccgcggatgggcactatttatatcaac

SEQ ID NO: 3 - DNA, 44 bp, Artificial Sequence
  Chemically synthesized
cgcggatcctaatacgactcactataggggccaccaacgacatt

SEQ ID NO: 4 - DNA, 20 bp, Artificial Sequence
  Chemically synthesized
ccggccttattccaagcggc

SEQ ID NO: 5 - DNA, 24 bp, Artificial Sequence
  Chemically synthesized
ctgactgctgtctacagtggcctg

SEQ ID NO: 6 - DNA, 24 bp, Artificial Sequence
  Chemically synthesized
agtcaggctccgggatccgtacag

SEQ ID NO: 7 - DNA, 50 bp, Artificial Sequence
  Chemically synthesized
ggggacaactttgtacaaaaaagttggcatgcctggctttacgtgctgcg

SEQ ID NO: 8 - DNA, 49 bp, Artificial Sequence
  Chemically synthesized
ggggacaactttgtacaagaaagttggtcacattccgtgcttcttgcgg

SEQ ID NO: 9 - DNA, 57 bp, Artificial Sequence
  Chemically synthesized
ggggacaactttgtacaagaaagttggttacctgcggcgggcggccgcggccccagc

SEQ ID NO: 10 - DNA, 51 bp, Artificial Sequence
  Chemically synthesized
gggacaactttgtacaaaaaagttggctcctcaccctctgcctccactgcc

SEQ ID NO: 11 - DNA, 24 bp, Artificial Sequence
  Chemically synthesized
gtcgcagccatgcctggctttacg

SEQ ID NO: 12 - DNA, 49 bp, Artificial Sequence
  Chemically synthesized
tcacttatcgtcgtcatccttgtaatccatgccgtgcttcttacggatg

SEQ ID NO: 13 - DNA, 40 bp, Artificial Sequence
  Chemically synthesized
atcaactgtatacaagcagctaggacagcaccctaatgtc

SEQ ID NO: 14 - DNA, 25 bp, Artificial Sequence
  Chemically synthesized
tgtattacaagctaggacagcacct

SEQ ID NO: 15 - DNA, 12 bp, Artificial Sequence
  Chemically synthesized
ggacagcaccct

SEQ ID NO: 16 - DNA, 13 bp, Artificial Sequence
  Chemically synthesized
caagctaggacag

SEQ ID NO: 17 - DNA, 26 bp, Artificial Sequence
  Chemically synthesized
tgtattacaagctaggacagcaccct

SEQ ID NO: 18 - DNA, 18 bp, Artificial Sequence
  Chemically synthesized
ggatccgtggttcccgtg

SEQ ID NO: 19 - DNA, 19 bp, Artificial Sequence
  Chemically synthesized
ccatggcaagcagacgatc

SEQ ID NO: 20 - DNA, 21 bp, Artificial Sequence
  Chemically synthesized
gtgggtcgctaaacctgagag

SEQ ID NO: 21 - DNA, 21 bp, Artificial Sequence
  Chemically synthesized
gttcaatgagttagctgtgtc

SEQ ID NO: 22 - DNA, 20 bp, Artificial Sequence
  Chemically synthesized
accacagtccatgccatcac

SEQ ID NO: 23 - DNA, 20 bp, Artificial Sequence
  Chemically synthesized
tccaccaccctgttgctgta

SEQ ID NO: 24 - protein, 15 aa, Artificial Sequence
  Chemically synthesized
VPGCYNNSHRDKALH

SEQ ID NO: 25 - protein, 15 aa, Artificial Sequence
  Chemically synthesized
TGSDHSYSLSSGTTE

SEQ ID NO: 26 - protein, 15 aa, Artificial Sequence
  Chemically synthesized
LMEVKMKEMKGSIRH

SEQ ID NO: 27 - DNA, 28 bp, Artificial Sequence
  Chemically synthesized
acaaagcttagtctcgcgatgctgccac

SEQ ID NO: 28 - DNA, 29 bp, Artificial Sequence
  Chemically synthesized
acagctagccgtaaagccaggcatggctg

SEQ ID NO: 29 - DNA, 20 bp, Artificial Sequence
  Chemically synthesized
cttaggacgagcttcatctg

SEQ ID NO: 30 - DNA, 21 bp, Artificial Sequence
  Chemically synthesized
gacgggatcaactccaagctg

SEQ ID NO: 31 - DNA, 38 bp, Artificial Sequence
  Chemically synthesized
acactcgagtagataggtattggcctatttgaaagaac

SEQ ID NO: 32 - DNA, 34 bp, Artificial Sequence
  Chemically synthesized
acacctaggtggcacatacctttaatcccagcac

SEQ ID NO: 33 - DNA, 26 bp, Artificial Sequence
  Chemically synthesized
agggcggccgcaagacctacacggtg

SEQ ID NO: 34 - DNA, 36 bp, Artificial Sequence
  Chemically synthesized
acacggccgatccccacatttcaaggacacttagct

SEQ ID NO: 35 - DNA, 38 bp, Artificial Sequence
  Chemically synthesized
acagcggccgctacctttcgcttaggacgagcttcatc

SEQ ID NO: 36 - DNA, 31 bp, Artificial Sequence
  Chemically synthesized
cttgcggccgccctgaaagtggacgctgcag

SEQ ID NO: 37 - DNA, 36 bp, Artificial Sequence
  Chemically synthesized
acaggcgcgccacgtctacgctaactctggcactgg

SEQ ID NO: 38 - DNA, 34 bp, Artificial Sequence
  Chemically synthesized
acaacgcgttgtattcgaatggacacgttatggc

SEQ ID NO: 39 - DNA, 39 bp, Artificial Sequence
  Chemically synthesized
acagctagcgccaccatgtccaatttactgaccgtacac

SEQ ID NO: 40 - DNA, 35 bp, Artificial Sequence
  Chemically synthesized
acacccgggagatcgccatcttccagcaggcgcac

SEQ ID NO: 41 - DNA, 24 bp, Artificial Sequence
  Chemically synthesized
gcctcagagctagaggctgctacg

SEQ ID NO: 42 - DNA, 24 bp, Artificial Sequence
  Chemically synthesized
tggaaggagtcacgaattctgcag

SEQ ID NO: 43 - DNA, 24 bp, Artificial Sequence
  Chemically synthesized
ggcgttctctttggaaaggtgttc

SEQ ID NO: 44 - DNA, 20 bp, Artificial Sequence
  Chemically synthesized
ctcgaaccacatccttctct

SEQ ID NO: 45 - DNA, 20 bp, Artificial Sequence
  Chemically synthesized
ccagtggagtatcccagcat

SEQ ID NO: 46 - DNA, 20 bp, Artificial Sequence
  Chemically synthesized
gaagttatggagcggagcag

SEQ ID NO: 47 - DNA, 24 bp, Artificial Sequence
  Chemically synthesized
atagcagtttccagtggagccctt

SEQ ID NO: 48 - DNA, 24 bp, Artificial Sequence
  Chemically synthesized
tggatcgcggacgcataggtatta

SEQ ID NO: 49 - DNA, 20 bp, Artificial Sequence
  Chemically synthesized
ttacttcccgccagctcttc

SEQ ID NO: 50 - DNA, 24 bp, Artificial Sequence
  Chemically synthesized
tgatgcattttgggggtatctctc

SEQ ID NO: 51 - DNA, 18 bp, Artificial Sequence
  Chemically synthesized
tcgtattccaacaggagg

SEQ ID NO: 52 - DNA, 18 bp, Artificial Sequence
  Chemically synthesized
aggcttttgcttcaccag

SEQ ID NO: 53 - DNA, 20 bp, Artificial Sequence
  Chemically synthesized
atgccaaagaaagaaacgac

SEQ ID NO: 54 - DNA, 20 bp, Artificial Sequence
  Chemically synthesized
agaggctgtagaacatgatt

SEQ ID NO: 55 - DNA, 23 bp, Artificial Sequence
  Chemically synthesized
ggcccagagcaagagaggtatcc

SEQ ID NO: 56 - DNA, 21 bp, Artificial Sequence
  Chemically synthesized
acgcacgatttccctctcagc

SEQ ID NO: 57 - DNA, 20 bp, Artificial Sequence
  Chemically synthesized
accacagtccatgccatcac

SEQ ID NO: 58 - DNA, 20 bp, Artificial Sequence
  Chemically synthesized
tccaccaccctgttgctgta

SEQ ID NO: 59 - DNA, 24 bp, Artificial Sequence
  Chemically synthesized
agtcaggctccgggatccgtacag

SEQ ID NO: 60 - DNA, 20 bp, Artificial Sequence
  Chemically synthesized
ccggccttattccaagcggc

SEQ ID NO: 61 - DNA, 18 bp, Artificial Sequence
  Chemically synthesized
gcagaccagatgctggaa

SEQ ID NO: 62 - DNA, 20 bp, Artificial Sequence
  Chemically synthesized
ttttctccggtcctgatgtc

SEQ ID NO: 63 - DNA, 19 bp, Artificial Sequence
  Chemically synthesized
gccacacacacacccttgt

SEQ ID NO: 64 - DNA, 20 bp, Artificial Sequence
  Chemically synthesized
aaggcaaggcatcctgacta

SEQ ID NO: 65 - DNA, 20 bp, Artificial Sequence
  Chemically synthesized
ggatggcatactgtggacct

SEQ ID NO: 66 - DNA, 20 bp, Artificial Sequence
  Chemically synthesized
agttgctttccactcgtgct

SEQ ID NO: 67 - DNA, 20 bp, Artificial Sequence
  Chemically synthesized
ccagtggagtatcccagcat

SEQ ID NO: 68 - DNA, 20 bp, Artificial Sequence
  Chemically synthesized
gaagttatggagcggagcag

SEQ ID NO: 69 - protein, 12 aa, Artificial Sequence
  Chemically synthesized
YYACAWRTCCCT

SEQ ID NO: 70 - protein, 13 aa, Artificial Sequence
  Chemically synthesized
ACTAYRNNNCCCR

(19) United States
(12) Patent Application Publication
(10) Pub. No.: US 2011/0201109 A1
(43) Pub. Date: Aug. 18, 2011

(54) Ronin is Essential for Perpetuity of Mouse ES Cells, and Acts Independently of Canonical Pathways

(75) Inventors: Thomas P. Zwaka, Houston, TX (US); Marion Dejosez, Houston, TX (US); Laura Jo Zitur, Dane, WI (US); Joshua Krumenacker, Summerfield, FL (US)

(73) Assignee: BAYLOR COLLEGE OF MEDICINE

(21) Appl. No.: 12/989,924

(22) PCT Filed: May 13, 2009
(86) PCT No.: PCT/US09/43827

§ 371 Date: Apr. 18, 2011

(30) Foreign Application Priority Data
May 13, 2008 (US) 61/127400

(51) Int. Cl. C12N 5/0735

(52) U.S. Cl. 435/366; 435/375; 435/377; 435/325

(57) ABSTRACT

The present invention, therefore, encompasses compositions and methods comprising Ronin, a Ronin activator, and methods of their use for maintaining the perpetuity of an ES cell phenotype.

### BACKGROUND OF THE INVENTION

Although a number of regulatory mechanisms specific to embryonic stem (ES) cells have been described, the full transcriptional circuitry underlying the control of ES cell survival and differentiation has not yet emerged. It has been proposed that a small core set of regulatory factors, mainly Oct4, Sox2, Nanog and Tcf3, whose targets include protein-coding and microRNA genes linked to the regulation of organismal and cellular development, maintain ES cells in a pluripotent state by acting through a regulatory circuit replete with autoregulatory feed-forward and other regulatory motifs (Boyer et al., 2005, Cell 122:947-956; Chen et al., 2008, Cold Spring Harbor Symposia on Quantitative Biol.; Cole et al., 2008, Genes Dev. 22:746-755; Loh et al., 2006, Nature Genetics 38:431-440; Marson et al., 2008, Cell 134:521-533). Added control is achieved through epigenetic mechanisms that operate in concert with Oct4 and other canonical pluripotency factors to modulate the transcription of genes required for ES cell differentiation (Bernstein et al., 2006, Cell 125:315-326; Boyer et al., 2006, Nature 441:349-353; Klochendler-Yeivin et al., 2000, EMBO Rep. 1:500-506; Lee et al., 2006, Cell 125:301-313). Thus, it is not surprising that transcriptional repression of key developmental pathways has been a dominant theme of research on ES cell pluripotency, with much less attention paid to factors that could actively sustain the pluripotent state.

Growing evidence supports the concept that perpetuation of the ES cell phenotype involves factors other than those controlling the cell cycle and differentiation. Ablation of the orphan nuclear receptor gene Gcnf, whose product suppresses numerous canonical pluripotency factors upon induction of differentiation, provides a striking example. Despite their continued upregulation of key pluripotency factors, Gcnf −/− ES cells were still able to differentiate, both in vitro and in the gastrulating embryo (Fuhrmann et al., 2001, Dev. Cell 1:377-387; Gu et al., 2005, Mol. Cell. Biol. 25:8507-8519). Furthermore, many ES and embryonic carcinoma cell lines, as well as induced pluripotent stem (iPS) cell lines, can fully execute a self-renewal program without being pluripotent (Mikkelsen et al., 2008, Nature 454:49-55; Takahashi and Yamanaka, 2006, Cell 126:663-676). Intriguingly, the self-renewal capacity of ES and iPS cells appears not to rely on the instructive signals typically controlling cell behavior pathways. For instance, impeding the extracellular signal-regulated kinase (ERK) pathway failed to block self-renewal, even in the absence of cytokines routinely used to support such growth (Ying et al., 2008, Nature 453:519-523), and in fact promoted restoration of the pluripotent state in somatic cells (Silva et al., 2008, Cell 132:532-536). However, these positive effects on self-renewal required concomitant inhibition of glycogen synthase kinase-3, whose activity otherwise compromised cell growth and viability. Antithetical roles of signal transduction pathways and cell metabolic capacity have similarly been observed in other eukaryotes, including the yeast Saccharomyces cerevisiae. In this system “expression capacity”, defined as the capacity of cells to produce energy and ribosomes in order to synthesize protein from genes, was negatively correlated with “pathway capacity”, defined as the ability to transduce signals (Colman-Lerner et al., 2005, Nature 437:699-706). On the strength of these and similar observations, pluripotency has been characterized as a constitutive replicative state, or “ground state”, of cellular identity in which cell intrinsic constituents, such as biosynthetic capacity and metabolic rate, directly govern the probability that pluripotent cells will self-renew (Ying et al., 2008, Nature 453:519-523). Ronin's regulatory functions appear to differ from those of canonical factors (Dejosez et al., 2008, Cell 133:1162-1174).

There has been a long standing need in the art to identify a means of sustaining self-renewing ES cells independently of canonical pluripotency factors. The present invention fulfills this need.

### SUMMARY OF THE INVENTION

One embodiment of the invention describes a method of maintaining the self-renewal capacity of an embryonic stem (ES) cell, the method comprising contacting the cell with an effective amount of Ronin or a Ronin activator, wherein when Ronin or Ronin activator contacts the ES cell, Ronin or Ronin activator maintain the self-renewal capacity of the ES cell. In one aspect, Ronin or Ronin activator is provided to the ES cell exogenously. In another aspect, the activator is selected from the group consisting of a protein, a nucleic acid, and a small molecule. In still another aspect, Ronin or a Ronin activator is expressed by the ES cell. In yet another aspect, the expression of the Ronin activator is controlled by a promoter. In still another aspect, the promoter is an inducible promoter. In another aspect, the ES cell is a mammalian embryonic stem cell. In still another aspect, the ES cell is a human embryonic stem cell.

Another embodiment of the invention includes a method of maintaining self-renewal capacity of an ES cell, the method comprising regulating expression of Ronin in the cell by an inducible promoter, wherein when Ronin expression is increased, the ES cell does not differentiate. In one aspect, the ES cell is a mammalian embryonic stem cell. In another aspect, the ES cell is a human embryonic stem cell.

Another embodiment of the invention describes a method of reversibly preventing the differentiation of an ES cell, the method comprising the steps of conditionally expressing Ronin or a Ronin activator in the ES cell, wherein the conditional expression of Ronin or a Ronin activator is under the control of an inducible promoter; contacting the ES cell with an inducing agent, wherein when the inducing agent contacts the ES cell, the inducing agent activates the inducible promoter, thereby increasing expression of Ronin or Ronin activator in the ES cell, wherein the ES cell does not differentiate; and, optionally, removing the inducing agent, whereby Ronin or Ronin activator expression is reduced in the ES cell, and the ES cell differentiates. In one aspect, the expression of Ronin is controlled by an inducible promoter. In another aspect, the cell is a mammalian embryonic stem cell. In still another aspect, the cell is a human embryonic stem cell.

Another embodiment of the invention describes a composition comprising an ES cell having an inducible promoter, wherein the promoter regulates the expression of Ronin or a Ronin activator by the ES cell. In one aspect the activator is selected from the group consisting of a protein, a nucleic acid, and a small molecule. In another aspect, the cell is a mammalian embryonic stem cell. In still another aspect, the cell is a human embryonic stem cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1I, is a series of images depicting Ronin, a nuclear THAP domain protein, whose expression is restricted to early embryonic cells and undifferentiated ES cells. FIG. 1A is a schematic diagram of the Ronin protein showing a THAP domain at the N-terminus. NLS, nuclear translocation signal; polyQ, polyglutamine tract; polyA, polyalanine sequence; THAP, Thanatos-associated protein; aa, amino acid. FIG. 1B is an image depicting SELEX identification of the consensus sequence recognized by Ronin DNA-binding motif (top) and gel-shift experiments with a specific DNA sequence (bottom). Triangles indicate gel shift complexes. FIG. 1C is an image depicting Northern Blot analysis of multiple mouse tissues. Positive control is mouse ES cell line R1. FIG. 1D is a pair of photomicrographs depicting X-Gal staining of tissues isolated from mpRonin-lacZ reporter mice in oocytes (left) and in some areas of the adult brain (right). FIG. 1E is a pair of photomicrographs depicting immunostaining of oocytes (left) and zygotes (right) isolated from adult females using a Ronin antiserum (bar=10 μm) FIG. 1F is a series of images depicting Ronin promoter-driven lacZ expression detected at the 2-cell stage of embryo development, the 8-cell stage, compact morula stage, and the blastocyst stage (E3.5). FIG. 1G is a series of images depicting immunostaining of Ronin in morula and blastocyst stage embryos and in in vitro inner cell mass (ICM) outgrowth. FIG. 1H is a pair of images depicting immunostaining of endogenous Ronin in mouse (left) and human ES cells (right) (bar=10 μm). FIG. 1I is a series of images depicting lacZ expression detected in undifferentiated ES cells grown in the presence of MEFs. No staining was detected after differentiation in the absence of MEFs and LIF or after EB formation for 3 days. (scale bars: D left 30 μm, D right 80 μm, E left 30 μm, right 20 μm.

FIG. 2, comprising FIG. 2A through FIG. 2D, is a series of images depicting Ronin's essential role in normal embryogenesis and for ES cells survival. FIG. 2A is an image depicting H&E staining of mouse uterine sections following Ronin^{+/-} crosses. Empty swollen decidua (marked by an asterisk, bar=1 cm, left) were similar in size to those containing wild-type embryos (middle), but displayed residual resorbed embryonic tissue (right) at day E6.5. FIG. 2B is a series of phase contrast images of blastocysts and ICM outgrowth (bar=30 μm). FIG. 2C is a pair of immunofluorescent images of Cre-GFP transfected ES cells. (bar=20 μm). FIG. 2D is a pair of immunofluorescent images depicting MEFs isolated from a Ronin^{flox/flox} animal.

FIG. 3, comprising FIG. 3A through FIG. 3E, is a series of images depicting the inhibitory effect of forced Ronin expression ES cell differentiation. FIG. 3A is a series of phase contrast images of control ES cells and EF1α-Ronin ES cells, stably overexpressing Ronin, (bar=20 μm) FIG. 3B is a series of images depicting alkaline phosphatase staining of cells treated as in FIG. 3A at two different magnifications (bar 10×=80 μm, bar 20×=40 μm). FIG. 3C is a graph depicting quantification of 3×50 colonies from the experiment described in FIG. 3B. The values are means and standard deviations from triplicate experiments. FIG. 3D is a photograph depicting a chimeric mouse generated by injection of EF1α-DRonin ES cells into blastocysts. FIG. 3E is an image depicting Western blot analysis of Stat3 and Phospho-Stat3 indicating comparing the amount and phosphorylation level of wild-type and Ronin-overexpressing ES cells after LIF removal.

FIG. 4, comprising FIG. 4A through FIG. 4F, is a series of images depicting the effect of Ronin on differentiation independent of canonical pluripotency factors. FIG. 4A is an image depicting semiquantitative RT-PCR analysis of pluripotency factors and marker genes for all three germ layers. FIG. 4B is a pair of graphs depicting the effect of siRNA against Oct4 and Ronin transfected into ES cells. The expression of their mRNA determined at the indicated time points by real-time quantitative PCR. Data are reported as means and standard deviations of triplicate experiments. FIG. 4C is a photomicrograph depicting alkaline phosphatase staining of ES cells after siRNA knockdown of Oct4. FIG. 4D is a graph depicting quantification of result in FIG. 4C. Bars represent means and standard deviations of triplicate experiments. FIG. 4E is a photomicrograph depicting ZBHTC4.1 (EF1α) ES cells (control) induced to differentiate by addition of doxycycline or ZBHTC4.1 (EF1α-Ronin) ES cells ectopically expressing Ronin exposed to doxycyclin. FIG. 4F is a graph depicting the quantification of FIG. 4E.

FIG. 5, comprising FIG. 5A and FIG. 5B, is a series of images depicting tumorigenic effect of ectopic expression of Ronin. FIG. 5A is a series of images depicting immunohistochemical staining of Oct4 in teratocarcinoma sections; bar (top)=0.5 cm, bar (bottom)=0.1 mm FIG. 5B is a series of images depicting H&E staining of EF1α-Ronin teratocarcinoma sections, bar=50 μm.

FIG. 6, comprising FIG. 6A through FIG. 6E, is a series of images depicting Ronin's effect as a transcriptional repressor and epigenetic modulator. FIG. 6A, left is a graph depicting box plots showing results of microarray analysis of control ES cells and ES cells 24 hours after transfection with pEF1α-hRonin-Flag. Each box represents median and 75th and 25th percentile values. FIG. 6B is a series of images depicting confocal images of 5-fluorouridine (5-FU) staining of newly transcribed RNA after induction of Ronin expression in a Ronin-inducible cell line, bar=10 μm. FIG. 6C is a graph depicting quantification of 3H-Uridine incorporation into newly transcribed RNA of control A172loxP cells and A172LPRonin-Flag cells after induction with 1 μg/ml doxycycline. Bars show means (and standard deviations) of triplicate experiments. FIG. 6D is an image depicting Western blot analysis of H3K9me2 methylation in the presence of induced Ronin expression by doxycycline. FIG. 6E is an image depicting chromatin immunoprecipitation and PCR of genomic regions containing the 3× sequence upstream of Gata4 and Gata6.

FIG. 7, comprising FIG. 7A through FIG. 7E, is a series of images depicting Ronin binding directly to HCF-1 and its association with a very large protein complex. FIG. 7A is an image depicting directional yeast two-hybrid approach to detect direct interaction of Ronin, Ronin-C and Ronin-N with candidate interacting proteins identified by mass spectrometry. Direct interaction of Ronin-C with HCF-1 is represented by growth of cotransfected MAV103 yeast on 50 mM 3AT-containing plates (AD=activation domain, DB=DNA binding domain). FIG. 7B is an image comparing the similar phenotypes obtained when HCF-1 was downregulated by siRNA in control ES cells (top) and Ronin ectopically expressing ES cells (bottom). FIG. 7C is an image depicting anti-Ronin-Flag Western blot of glycerol gradient fractions of EF1α-Ronin ES cell nuclear extracts purified with wheat germ agglutinin beads. Ronin is present in the first 5 fractions and in fractions 20 to 23 with a peak in 20. Comparison with the elution peaks of protein standards with known sizes is shown on top. FIG. 7D is an image depicting Western blot analysis of Myc-tagged proteins after GST immunoprecipitation. R, Ronin; C, Ronin C-terminus; N, Ronin N-terminus; D, empty destination vector. FIG. 7E is a schematic diagram illustrating a proposed model for the mechanism of Ronin function.

FIG. 8, comprising FIG. 8A through FIG. 8E, is a series of images depicting Ronin binding to a highly conserved promoter element. FIG. 8A is a group of graphs depicting ChIP-seq results for four representative genes bound by Ronin. Relative binding positions across particular chromosomal regions (x-axis, top of each region) and the first exons (bottom of each region) are shown for Nbr1 (neighbor of Brca1 gene 1), RpoI-2 (RNA polymerase I), Rangap1 (RAN GTPase activating protein 1) and Prcc (papillary renal cell carcinoma [translocation-associated]). The y-axis indicates fold enrichment of Ronin protein binding in the proximal promoter region (p<10-9 for all genes). FIG. 8B is a graph depicting the distance of each Ronin binding event from the transcriptional start site (TSS). FIG. 8C is an image depicting identification of the consensus Ronin binding motif (RBM) depicted as a bit matrix (bottom). FIG. 8D is an image depicting EMSA analysis of newly identified Ronin binding sequence. ES cell extracts show a specific shift (S) that supershifts when Ronin antiserum is added. FIG. 8E is an image depicting verification of ChIP-seq results by ChIP-PCR analysis.

FIG. 9, comprising FIG. 9A through FIG. 9F, is a series of images depicting Ronin and canonical pluripotency factors control distinct genetic programs. FIG. 9A depicts ChIP-seq results obtained by precipitation with antibodies against Ronin, Oct4, Sox2, Nanog and Tcf3. FIG. 9B depicts the results of cluster analysis of genes bound by the indicated factors and reveals that Ronin does not cluster with canonical pluripotency factors Oct4, Nanog and Sox2. FIG. 9C is a graph depicting overlap between Ronin binding sites and H3K4me3, Suz12, H3K79me2 and H3K36me3. FIG. 9D is a pair of graphs that depicts GSEA analysis of Ronin target genes. Top panel: Significant enrichment of Ronin target genes in ES cells compared with differentiated cells. Bottom panel: Significant enrichment of Ronin target genes in Ronin-overexpressing cell line by comparison with wild-type ES cells indicates that Ronin positively controls its target genes. FIG. 9E depicts expression of Ronin target genes during the differentiation of mouse ES cells. FIG. 9F is a graph depicting PANTHER biological pathway analysis of Ronin target genes. Fold enrichment is shown on the yaxis.

FIG. 10, comprising FIG. 10A through FIG. 10D, is a series of images depicting that Hcf-1 interacts with Ronin and mediates its function in mouse ES cells. FIG. 10A depicts ChIP-seq results obtained in R1 mouse ES cells after immunoprecipitation with Ronin and Hcf-1 antibodies. A representative region on mouse chromosome 8D3, containing three Ronin-bound genes (Cenpt [G630055Po3Rik], Ronin [Thap11] and Nutf2) is shown. FIG. 10B depicts Ronin structure and position of Hcf-1 binding motif located towards the C-terminus (top), which is highly conserved throughout the animal kingdom (bottom). FIG. 10C depicts the results of a directional yeast two-hybrid assay in which the Ronin C-terminus (Ronin-C) binds to the N-terminus of Hcf-1 (N-Hcf1) as demonstrated by growth on 50 mM 3AT (middle); whereas mutation of the Hcf-1 binding motif from DHSY to DHSA (RoninDSHA-C) completely abolishes binding to Hcf-1 (bottom). FIG. 10D is a graph depicting quantification of experiment shown in FIG. 10E. Values are means of ±SD of triplicate experiments. FIG. 10E is a series of images depicting the morphology of control, Ronin-overexpressing and Ronin-DSHA-overexpressing mouse ES cells after 3 days of culture in the presence of Lif (top panels, 100,000 cells/10 cm2, 10× magnification). Cells (10,000 cells/10 cm2) were stained for alkaline phosphatase activity after 4 days of culture in the absence of Lif (bottom panels; 20× magnification).

FIG. 11, comprising FIG. 11A through FIG. 11E, is a series of images depicting how Ronin regulates ES cell growth, metabolism and DNA protection. FIG. 11A, Top panel: Average diameter of control, Ronin- or Ronin^{DSHA}-overexpressing mouse ES cells. Values are means of ±SD of triplicate experiments. Asterisk indicates a significant difference of Ronin-overexressing cells from control or Ronin^{DSHA}-overexpressing cells (p=5.63×10⁻⁴). Bottom panel: Pellet size of 16×10⁶ control, Ronin- or Ronin^{DSHA}-overexpressing mouse ES cells after resuspension in PBS and centrifugation. FIG. 11B, Top panel: Protein amount per cell in lines shown in FIG. 4A indicating significantly greater protein amounts in Ronin-overexpressing cells compared with Ronin^{DSHA} or control cells (Asterisk indicates p=1.6410×10⁻², values are means of ±SD of triplicate experiments). FIG. 11B, Bottom panel: Determination of newly synthesized protein. Control cells (open arrow head), Ronin-overexpressing (filled arrow head) and Ronin^{DSHA}-overexpressing cells (grey solid line) stained with AHA-Alexa Fluor488; additional controls were treated with cycloheximide (black dotted line) to block protein synthesis or were not labeled with AHA (grey dotted line). FIG. 11C is a graph depicting ATP content of control, Ronin- and Ronin^{DSHA}-overexpressing cells, represented by relative luminescence units (RLUx10⁶/5×10⁴ cells) (Asterisk indicates p=3.92×10⁻³, values are means of ±SD of triplicate experiments). FIG. 11D is a series of graphs depicting cell cycle analysis of control (left), Ronin-(middle) and Ronin^{DSHA}-overexpressing (right) cells by BrdU/7-AAD staining. FIG. 4E is a series of images depicting immunofluorescence of control (left), Ronin-(middle) and Ronin^{DSHA}-overexpressing cells (right) with use of antibody staining against H2AX without prior treatment (top panels) or induction of DNA damage by H₂O₂ (bottom panels) (20× magnification).

FIG. 12, comprising FIG. 12A through FIG. 12H, depicts Ronin reinstatement of a self-renewal program without activating the canonical pluripotency network. FIG. 5A depicts immunofluorescence staining for Ronin expression during reprogramming. FIG. 12B depicts Ronin knockout MEFs do not form clusters of ES cells and do not show AP-positive colonies at 16 days after iPS induction with Oct4/Sox2/Klf4/c-Myc. All expanded colonies are heterozygous for Ronin. FIG. 12C depicts transduction of MEFs with Ronin together with c-Myc, Klf4 and Sox2 (RMKS) leads to formation of iSC (induced self-renewing cells). FIG. 12D depicts phase contrast of representative colonies of one cell line established from MEF by delivery of Ronin, c-Myc, Klf4 and Sox2 (RMKS), showing typical ES cell morphology; O=Oct4 (10× magnification). FIG. 12E depicts members of the canonical pluripotency network are not activated by Ronin during iSC induction. FIG. 12F depicts hematoxylin and eosin staining of tumor tissue section after injection of iSCs induced with Ronin, c-Myc and Klf4 into immunocompromised mice (20× magnification). FIG. 12G depicts microarray analysis of an iSC cell line and MEFs, demonstrating initiation of a gene expression signature differing from that of MEFs, FIG. 12H depicts ES cells were highly enriched for a gene expression signature.

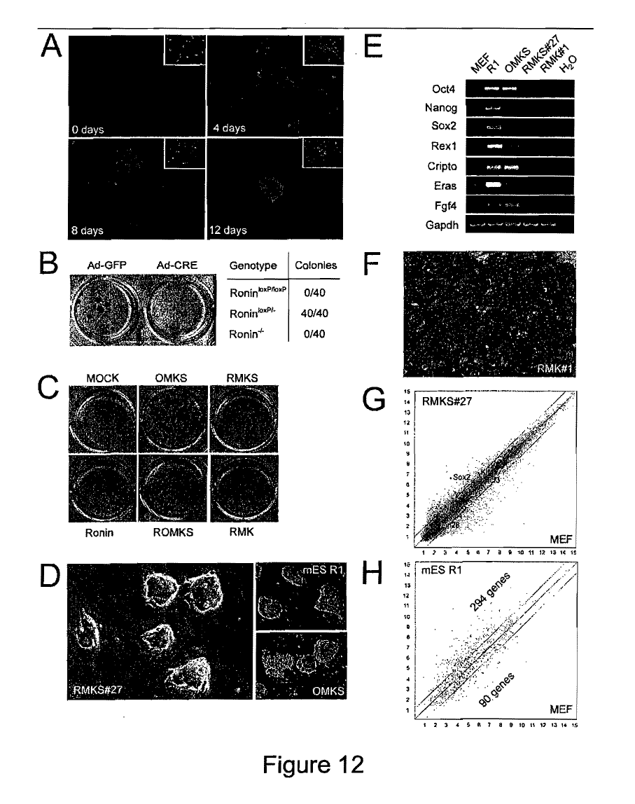

FIG. 13 is a schematic illustration depicting a proposed model of Ronin-controlled biological processes and autoregulation in ES cells. The processes regulated by Ronin directly support the self-renewal of ES cells, while those responding to Oct4, Sox2, Nanog and Tcf3 participate by repressing key developmental pathways. Thin black lines indicate a weak association.

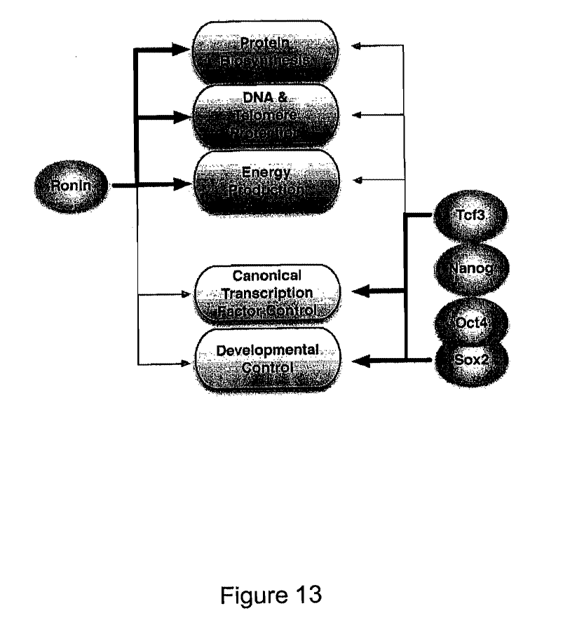

### DETAILED DESCRIPTION OF THE INVENTION

The present invention is based, in part, on the discovery of a novel pluripotency factor, Ronin, that is associated with sequence-specific DNA binding and epigenetic silencing of gene expression.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The phrase “activator,” as used herein, means to increase a molecule, a reaction, an interaction, a gene, an mRNA, and/or a protein's expression, stability, function or activity by a measurable amount. Activators are compounds that, e.g., bind to, partially or totally stimulate, increase, promote, increase activation, activate, sensitize, or up-regulate a protein, a gene, an mRNA stability, expression, function and activity. A “Ronin activator,” as used herein, may refer to a Ronin protein or fragment thereof, a nucleic acid that encodes Ronin, or any compound, gene, molecule, or agent that increases Ronin's expression, activity, stability, or function.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The phrase “inhibit,” as used herein, means to reduce a molecule, a reaction, an interaction, a gene, an mRNA, and/or a protein's expression, stability, function or activity by a measurable amount or to prevent its expression, stability, function, or activity entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “stem cell,” as used herein, refers to a pluripotent or lineage-uncommitted cell which is potentially capable of an unlimited number of mitotic divisions to either renew itself or to produce progeny cells, and which is able to differentiate into multiple cell types derived from single-cell clones.

The term “pluripotent” or “pluripotency,” as used herein, refers to a cell that can differentiate into all cell types except for extra-embryonic tissue, in contrast to a “totipotent cell,” which can produce every cell type including extraembryonic tissue. A “pluripotent cell” or a “pluripotent stem cell,” as used herein, is a cell that has the potential to differentiate into any of the three germ layers: endoderm, mesoderm, or ectoderm. Accordingly, a “pluripotent cell” or “a pluripotent stem cell” can give rise to any fetal or adult cell type.

The term “maintaining the pluripotency of a cell” or “maintaining the pluripotency of an embryonic stem cell,” as used herein refers to any means, process, or method whereby the potential of a pluripotent cell to differentiate into any of the three germ layers (endoderm, mesoderm, or ectoderm) is preserved, maintained, prolonged, or extended. Alternatively, the terms “maintaining the pluripotency of a cell” or “maintaining the pluripotency of an embryonic stem cell,” as used herein, may also refer to any means, process, or method whereby the differentiation of a pluripotent cell is inhibited.

The term “maintaining the self-renewing capacity of a cell” or “maintaining the self-renewing capacity of an embryonic stem cell,” as used herein refers to any means, process, or method whereby self-renewal of a pluripotent cell is preserved, unimpeded, perpetuated, sustained, maintained, prolonged, or extended. The phrase also encompasses perpetuating an embryonic stem cell phenotype, or sustaining a cell in a pluripotent state.

An “isolated cell” refers to a cell which has been separated from other components and/or cells which naturally accompany the cell in a tissue or mammal.

As used herein, a “substantially purified cell” is a cell that has been purified from other cell types with which it is normally associated in its naturally-occurring state.

“Expandability” is used herein to refer to the capacity of a cell to proliferate, for example, to expand in number or, in the case of a population of cells, to undergo population doublings. Cells are expanded in culture when they are placed in a growth medium under conditions that facilitate cell growth and/or division, resulting in a larger population of the cells.

As used herein, the term “growth medium” is meant to refer to a culture medium that promotes proliferation of cells.

“Proliferation” is used herein to refer to the reproduction or multiplication of similar forms, especially of cells. That is, proliferation encompasses production of a greater number of cells, and can be measured by, among other things, simply counting the numbers of cells, and the like. The rate of cell proliferation is typically measured by the amount of time required for the cells to double in number, otherwise known as the doubling time.

As used herein, “cell culture” refers to the process whereby cells, taken from a living organism, are grown under defined conditions.

A “primary cell culture” refers to a culture of cells, tissues or organs taken directly from an organism.

As used herein, “subculture” refers to the transfer of cells from one growth container to another growth container.

“Exogenous” refers to any material introduced into or produced outside an organism, cell, or system.

As used herein, the term “phenotype” or “phenotypic characteristics” should be construed to mean the expression of a specific biomarker protein or nucleic acid, or a combination of biomarker proteins or nucleic acids that distinguishes one cell from another.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

The term “oligonucleotide” typically refers to short polynucleotides.

It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DESCRIPTION

The present invention is related to the discovery of a novel perpetuity factor, Ronin. Ronin controls a unique genetic program that is activated to ensure unimpeded self-renewal of ES cells without inducing differentiation or activation of canonical pluripotency factors. Thus, Ronin may modulate or activate expression of multiple genes that are either directly or indirectly involved in self-renewal of ES cells. The present invention, therefore, encompasses compositions and methods comprising Ronin, a Ronin activator, and methods of their use for maintaining the perpetuity of an ES cell phenotype.

### I. Compositions

Accordingly, the present invention provides compositions comprising Ronin and Ronin activators useful in the practice of the methods of the present invention.

### A. Ronin

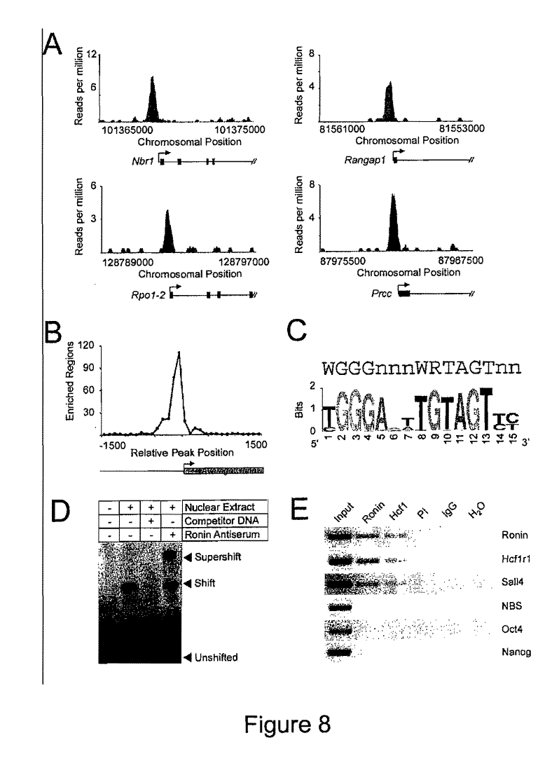

Ronin is a protein containing a THAP domain which comprises a zinc-finger DNA-binding motif defined, in part, by a C₂CH signature (Cys-Xaa₂₋₄-Cys-Xaa₃₃-50-Cys-X-aa2). The DNA sequence most often targeted by Ronin matches the highly conserved M4 promoter sequence, which previously lacked any known binding factors (Xie et al., 2005). An alignment of protein sequences for Ronin obtained from a variety of species is depicted in FIG. 8B and demonstrated that Ronin is a highly conserved protein.

Functionally, the gene set controlled by Ronin in ES cells is devoid of genes associated with development, but rather contains genes involved in protein biosynthesis, energy production, telomere preservation and the DNA damage response.

### B. Ronin Activators

The present invention encompasses various Ronin activators. An activator may be a protein, a peptide, a nucleic acid, a molecule, compound, or other chemical or biological agent that, e.g., bind to, partially or totally stimulate, increase, promote, increase activation, activate, sensitize, or up-regulate a protein, a gene, or mRNA stability, expression, function, or activity.

Accordingly, a “Ronin activator,” as used herein, refers to any nucleic acid, protein, peptide, molecule, compound or agent that partially or totally stimulates, increases, promotes, increases activation, activates, sensitizes, or up-regulates a protein, a gene, or mRNA stability, expression, function, or activity of Ronin.

In one embodiment, a Ronin activator is a Ronin protein molecule, or a fragment thereof. In another embodiment, a Ronin activator is a nucleic acid that encodes Ronin or a fragment thereof. In still another embodiment, a Ronin activator is a nucleic acid that encodes an upstream regulator or downstream effector of Ronin, such that the expression, activity, or stability of Ronin is increased or enhanced in an ES cell. In yet another embodiment, a Ronin activator is any compound, molecule, biological agent or chemical agent that increases Ronin's expression, activity, stability, or function in an ES cell.

Methods of obtaining Ronin activators are known in the art and include chemical organic synthesis or biological means. Biological means includes purification from a biological source, recombinant synthesis and in vitro translation systems, using methods well known in the art.

B1. Ronin activators-Protein

When the Ronin activator is a protein or a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxycarbonyl as the α-amino protecting group, and the FMOC method, which utilizes 9-fluorenylmethyloxycarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being affected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use in accordance with the invention, a peptide is purified to remove contaminants. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

The present invention also provides for analogs of polypeptides which comprise a Ronin activator sequence. Analogs may differ from naturally occurring proteins or polypeptides by conservative amino acid sequence differences or by modifications which do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups: (a) glycine, alanine; (b) valine, isoleucine, leucine; (c) aspartic acid, glutamic acid; (d) asparagine, glutamine; (e) serine, threonine; (f) lysine, arginine; (g) phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

The present invention should also be construed to encompass “mutants,” “derivatives,” and “variants” of the peptides of the invention (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as the Ronin polypeptides disclosed herein, in that the peptide has biological/biochemical properties. A biological property of the polypeptides of the present invention should be construed but not be limited to include, the ability of the polypeptide to be secreted when expressed in a host cell and the ability of the protein to inhibit virus infection. Further, the invention should be construed to include naturally occurring variants or recombinantly derived mutants of Ronin polypeptide sequences.

### B2. Ronin Activators—Nucleic Acids

When the Ronin activator comprises a nucleic acid, any number of procedures may be used for the generation of an isolated nucleic acid encoding the activator as well as derivative or variant forms of the isolated nucleic acid, using recombinant DNA methodology well known in the art (see Sambrook et al., MOLECULAR CLONING: A LABORATORY MANUAL, volumes 1-3, 3rd ed., Cold Spring Harbor Press, NY 2001) and by direct synthesis. For recombinant and in vitro transcription, DNA encoding RNA molecules can be obtained from known clones of Ronin, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al., MOLECULAR CLONING: A LABORATORY MANUAL, volumes 1-3 (3rd ed., Cold Spring Harbor Press, NY 2001).

An isolated nucleic acid of the present invention can be produced using conventional nucleic acid synthesis or by recombinant nucleic acid methods known in the art and described elsewhere herein (Sambrook et al., MOLECULAR CLONING: A LABORATORY MANUAL, volumes 1-3, 3rd ed., Cold Spring Harbor Press, NY 2001) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang et al., (1979, Meth. Enzymol., 68: 90) and U.S. Pat. No. 4,356,270.

In addition, the compositions of the present invention can be synthesized in whole or in part, or an isolated nucleic acid can be conjugated to another nucleic acid using organic synthesis such as the phosphodiester method, which has been used to prepare a tRNA gene. See Brown et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing nucleic acids, described in U.S. Pat. No. 4,293,652, is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention.

As nucleic acids are polymers of subunits or monomers, many of the modifications described below occur at a position which is repeated within a nucleic acid, e.g., a modification of a base, or a phosphate moiety, or a non-linking O of a phosphate moiety. In some cases the modification will occur at all of the subject positions in the nucleic acid but in many, and in fact in most cases it will not. By way of example, a modification may only occur at a 3′ or 5′ terminal position, in a terminal region, e.g., at a position on a terminal nucleotide, or in the last 2, 3, 4, 5, or 10 nucleotides of a strand. A component can be attached at the 3′ end, the 5′ end, or at an internal position, or at a combination of these positions. For example, the component can be at the 3′ end and the 5′ end; at the 3′ end and at one or more internal positions; at the 5′ end and at one or more internal positions; or at the 3′ end, the 5′ end, and at one or more internal positions. For example, a phosphorothioate modification at a non-linking O position may only occur at one or both termini, or may only occur in a terminal region, e.g., at a position on a terminal nucleotide or in the last 2, 3, 4, 5, or 10 nucleotides of the oligonucleotide. The 5′ end can be phosphorylated.

For increased nuclease resistance and/or binding affinity to the target, an oligonucleotide agent, can include, for example, T-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, O-AMINE and aminoalkoxy, O(CH₂), AMINE, (e.g., AMINE=NH₂; alkylamino, dialkylamino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

Preferred substitutents include but are not limited to 2′-methoxyethyl, 2′-OCH3,2′-O-allyl, 2′-C-allyl, and 2′-fluoro.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

One way to increase resistance is to identify cleavage sites and modify such sites to inhibit cleavage. For example, the dinucleotides 5′-UA-3′,5′-UG-3′,5′-CA-3′,5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. In certain embodiments, all the pyrimidines of the miRNA inhibitor carry a T-modification, and the miRNA inhibitor therefore has enhanced resistance to endonucleases.

In addition, to increase nuclease resistance, the 2′ modifications can be used in combination with one or more phosphate linker modifications (e.g., phosphorothioate). The so-called “chimeric” oligonucleotides are those that contain two or more different modifications.

With respect to phosphorothioate linkages that serve to increase protection against RNase activity, the miRNA inhibitor can include a phosphorothioate at least the first, second, or third internucleotide linkage at the 5′ or 3′ end of the nucleotide sequence. In one embodiment, the miRNA inhibitor includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In a preferred embodiment, the miRNA inhibitor includes at least one 2′-O-methyl-modified nucleotide, and in some embodiments, all of the nucleotides of the miRNA inhibitor include a 2′-O-methyl modification.

The 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

The oligonucleotide can be constructed using chemical synthesis and/or enzymatic ligation reactions using procedures known in the art. For example, an oligonucleotide can be chemically synthesized using naturally occurring nucleotides or variously modified nucleotides designed to increase the biological stability of the molecules or to increase the physical stability of the duplex formed between the oligonucleotide and target nucleic acids, e.g., phosphorothioate derivatives and acridine substituted nucleotides can be used. Other appropriate nucleic acid modifications are described herein. Alternatively, the oligonucleotide can be produced biologically using an expression vector into which a nucleic acid has been subcloned in an antisense orientation (i.e., RNA transcribed from the inserted nucleic acid will be of an antisense orientation to a target nucleic acid of interest (e.g., an mRNA, pre-mRNA, or an miRNA).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

Once a nucleic acid Ronin activator is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook et al., MOLECULAR CLONING: A LABORATORY MANUAL, volumes 1-3 (3rd ed., Cold Spring Harbor Press, NY 2001).

Once the nucleic acid for a Ronin polypeptide is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the Ronin polypeptides of the invention.

### Vectors

Ronin activators comprising a nucleic acid may be replicated in wide variety of cloning vectors in a wide variety of host cells.

In brief summary, the expression of natural or synthetic nucleic acids encoding a Ronin activator will typically be achieved by operably linking a nucleic acid encoding the Ronin activator or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In some aspects, the expression vector is selected from the group consisting of a viral vector, a bacterial vector, and a mammalian cell vector. Numerous expression vector systems exist that comprise at least a part or all of the compositions discussed above. Prokaryote- and/or eukaryote-vector based systems can be employed to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., MOLECULAR CLONING: A LABORATORY MANUAL, volumes 1-3 (3rd ed., Cold Spring Harbor Press, NY 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (e.g., WO 01/96584; WO 01/29058; and U.S. Pat. No. 6,326,193).

For expression of a Ronin activator or portions thereof; at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.”Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (e.g., U.S. Pat. No. 4,683,202, U.S. Pat. No. 5,928,906).

Naturally, it will be important to employ a promoter and/or enhancer that effectively directs the expression of the DNA segment in the cell type, organelle, and organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or polypeptides. The promoter may be heterologous or endogenous.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a Ronin activator, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### B3. Ronin Activators—Small Molecules

When the Ronin activator is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

### C. Embryonic Stem Cells

The present invention encompasses any embryonic stem cell or embryonic stem cell line, both known and heretofore as yet unknown. Methods for procuring, culturing, and maintaining an embryonic stem (ES) cell or an ES cell line of the present invention are well known in the art. Indeed, ES cells from various mammalian embryos have been successfully grown in the laboratory. Evans and Kaufman, 1981, (Nature 292:154-156) and Martin, 1981, (PNAS 72:1441-1445) showed that it is possible to derive permanent lines of embryonic cells directly from mouse blastocysts. Thomson successfully derived permanent cell lines from rhesus and marmoset monkeys (Thomson et al., 1996, Biol. Reprod. 55:254-259; Thomson et al., 1995, PNAS 92:7844-7848). Pluripotent cell lines have also been derived from pre-implantation embryos of several domestic and laboratory animal species such as bovines (Evans et al., 1990, Theriogenology 33:125-128) Porcine (Evans et al., 1990, Theriogenology 33:125-128; Notarianni et al., 1990 J. Reprod. Fertil. Suppl. 41:51-56), Sheep and goat (Meinecke-Tillmann and Meinecke, 1996, J. Animal Breeding and Genetics 113:413-426; Notarianni et al., 1991, J. Reprod. Fertil. Suppl. 43:255-260), rabbit (Giles et al., 1993, Mol. Reprod. Dev. 36:130-138; Graves et al., 1993, Mol. Reprod. Dev. 36:424-433) Mink (Sukoyan et al., 1992, Mol. Reprod. Dev. 33:418-431)) rat (Iannaccona et al., 1994, Dev. Biol. 163:288-292) and Hamster (Doetschman et al., 1988, Dev. Biol. 127:224-227). Recently, Thomson et al., 1998, (Science 282:1145-1147) and Reubinoff et al., 2000, (Nature Biotech. 18:299-304) have reported the derivation of human ES cell lines. These human ES cells resemble the rhesus monkey ES cell lines.

ES cells are derived from the ICM of the blastocyst, an early stage of the developing embryo. The blastocyst is the stage of embryonic development prior to implantation that contains two types of cells: trophectoderm, outer layer which gives rise to extra embryonic membranes, and the inner cell mass (ICM) which forms the embryo proper.

ES cells derived from the ICM during the blastocyst stage, however, can be cultured in the laboratory and under the right conditions proliferate indefinitely. ES cells growing in this undifferentiated state retain the potential to differentiate into cells of all three embryonic tissue layers. Ultimately, the cells of the inner cell mass give rise to all the embryonic tissues. It is at this stage of embryogenesis, that ES cells can be derived from the ICM of the blastocyst.

The ability to derive ES cells from blastocysts and grow them in culture seems to depend in large part on the integrity and condition of the blastocyst from which the cells are derived. In short, the blastocyst that is large and has distinct inner cell mass tends to yield ES cells most efficiently. Several methods have been used for isolation of inner cell mass (ICM) for the establishment of embryonic stem cell lines. The most common methods are as follows:

Natural Hatching of the Blastocyst In this procedure blastocyst is allowed to hatch naturally after plating on the feeder layer. The inner cell mass (ICM) of the hatched blastocyst develops an outgrowth. This outgrowth is removed mechanically and is subsequently grown for establishing embryonic stem cell lines.

Microsurgery Another method of isolation of inner cell mass is mechanical aspiration called microsurgery. In this process, the blastocyst is held by the holding pipette using micromanipulator system and positioned in such a way that the inner cell mass (ICM) is at 9 o'clock position. The inner cell mass (ICM) is aspirated using a bevel-shaped biopsy needle which is inserted into the blastocoel cavity. The operation at the cellular level requires tools with micrometer precision, thereby minimizing damage and contamination.

Immunosurgery Immunosurgery is a commonly used procedure to isolate inner cell mass (ICM). The inner cell mass (ICM) is isolated by complement mediated lysis. In this procedure, the blastocyst is exposed either to acid tyrode solution or pronase enzyme solution in order to remove the zona pellucida (shell) of blastocyst. The zona free embryo is then exposed to human surface antibody for about 30 min to one hour. This is followed by exposure of embryos to guinea pig complement in order to lyse the trophectoderm. The complement mediated lysed trophectoderm cells are removed from inner cell mass (ICM) by repeated mechanical pipetting with a finely drawn Pasteur pipette. All the embryonic stem cell lines reported currently in the literature have been derived by this method. However, this method has several disadvantages. First the embryo is exposed for a long time to acid tyrode or pronase causing deleterious effects on embryo, thereby reducing the viability of embryos. Second, it is a time consuming procedure as it takes about 1.5 to 2.0 hours. (Narula et al., 1996, Mol. Reprod. Dev. 44:343-351). Third, the yield of inner cell mass (ICM) per blastocyst is low. Fourth, critical storage conditions are required for antibody and complement used in the process. Last, it involves the risk of transmission of virus and bacteria of animal origin to humans, as animal derived antibodies and complement are used in the process. In this process, two animal sera are used. One is rabbit antihuman antiserum and the other is guinea pig complement sera. The human cell lines studied to date are mainly derived by using a method of immunosurgery, where animal based antisera and complement was used.

### D. Modified ES Cells

In one embodiment, the present invention contemplates ES cells expressing a Ronin activator, as taught herein. Accordingly, the invention further contemplates the ability to transduce embryonic stem cells and/or their differentiated progeny with particular nucleic acids, thereby giving rise to genetically modified stem cells and progeny. Transduction of human embryonic stem cell (hESC) lines is also taught in US Patent Application Publication No. 20050079616.

As used herein, “transduction of embryonic stem cells” refers to the process of transferring exogenous genetic material into an embryonic stem cell. The terms “transduction”, “transfection” and “transformation” are used interchangeably herein, and refer to the process of transferring exogenous genetic material into a cell. As used herein, “exogenous genetic material” refers to nucleic acids or oligonucleotides, either natural or synthetic, that are introduced into the cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include electroporation, transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization, proteins that confer intracellular localization and/or enhance intracellular half life, and the like. Polymeric delivery systems also have been used successfully to deliver nucleic acids into cells, as is known by those skilled in the art.

One method of introducing exogenous genetic material into cells is through the use of replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art. A preferred retroviral expression vector includes an exogenous promoter element to control transcription of the inserted exogenous gene. Such exogenous promoters include both constitutive and inducible promoters.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence which works with the coding sequence (in cis) to change the basal transcription level dictated by the promoter. Preferably, the exogenous genetic material is introduced into the genome immediately downstream from the promoter so that the promoter and coding sequence are operatively linked so as to permit transcription of the coding sequence.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., 1991, Proc. Natl. Acad. Sci. USA 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA 86: 10006-10010), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. By placing a recombinant gene under the control of an inducible promoter, the gene may be conditionally or reversibly expressed. “Conditionally expressed,” as used herein, refers to a gene that is only expressed when certain conditions are present, such as an inducing agent contacting an inducible promoter. Similarly, conditional expression of a gene under control of an inducible promoter may be reversible. That is, by way of a non-limiting example, when an inducing agent contacts an inducible promoter a gene is expressed, but when the inducing agent is removed, the gene is no longer expressed.

Thus, by selecting the appropriate promoter (constitutive versus inducible; strong versus weak), it is possible to control both the existence and level of expression of a Ronin activator in a genetically modified ES cell. Selection and optimization of these factors for delivery of an effective amount of a Ronin activator is deemed to be within the scope of one of ordinary skill in the art without undue experimentation, taking into account the above-disclosed factors.

In addition to at least one promoter and at least one heterologous nucleic acid encoding a Ronin activator, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The selection and optimization of a particular expression vector for expressing a specific gene product in a cell is accomplished by obtaining the gene, preferably with one or more appropriate control regions (e.g., promoter, insertion sequence); preparing a vector construct comprising the vector into which is inserted the gene; transfecting or transducing cultured cells in vitro with the vector construct; and determining whether the gene product is present in the cells.

Genes which affect regulation can also be administered, alone or in combination with a gene supplementing or replacing a specific function. For example, a gene encoding a protein which suppresses expression of a particular protein-encoding gene can be administered.

Accordingly, the invention includes an ES cell comprising a promoter that regulates expression of Ronin or a Ronin activator by the ES cell. As described elsewhere herein, a promoter of the invention may be either constitutively or conditionally active. When the promoter is constitutively active, the ES cell expresses Ronin or a Ronin activator all the time. When the promoter is conditionally active, the ES cell expresses Ronin or a Ronin activator only when an inducing agent in present.

E. Pharmaceutical compositions

The present invention includes a pharmaceutical composition comprising Ronin or a Ronin activator. In another embodiment, the present invention includes a pharmaceutical composition comprising an ES cell expressing Ronin. In still another embodiment, the present invention includes a composition comprising an ES cell expressing a Ronin activator. In one embodiment, the present invention includes a pharmaceutical composition comprising a substantially pure population of modified ES cells of the invention. Compositions comprising an ES cell can be incorporated into pharmaceutical compositions suitable for administration to a subject, preferably a human. The cells are preferably human cells.

As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. Supplementary active compounds can also be incorporated into the compositions.

A formulated composition comprising Ronin, a Ronin activator, or an ES cell expressing Ronin or a Ronin activator can assume a variety of states. Generally, the composition is formulated in a manner that is compatible with the intended method of administration.

A composition of the instant invention can be formulated in combination with another agent, e.g., another therapeutic agent or an agent that stabilizes the composition comprising Ronin, a Ronin activator, an ES cell expressing Ronin, or and ES cell expressing a Ronin activator.

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are distilled water, physiological saline, aqueous solutions of dextrose, and the like. For water soluble formulations, the pharmaceutical composition preferably includes a buffer such as a phosphate buffer, or other organic acid salt, preferably at a pH of between about 7 and 8. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences, e.g., Remington's Pharmaceutical Science, latest edition (Mack Publishing Company, Easton, Pa.).

An composition of the invention may be administered into a recipient in a wide variety of ways. Preferred modes of administration are parenteral, intravenous, intra-arterial, intramuscular, surgical implant, infusion pump, or via catheter.

### II. Methods

The present invention provides a method of maintaining the self-renewal capacity of an embryonic stem (ES) cell by contacting the ES cell with an effective amount of Ronin or a Ronin activator. It will be appreciated by the skilled artisan that Ronin or a Ronin activator may be brought into contact with an ES cell by administering an effective amount of exogenous Ronin or Ronin activator. In one embodiment, a Ronin activator is a Ronin protein molecule, or a fragment thereof. In another embodiment, a Ronin activator is a nucleic acid that encodes Ronin or a fragment thereof. In still another embodiment, a Ronin activator is a nucleic acid that encodes an upstream regulator or downstream effector of Ronin, such that the expression, activity, or stability of Ronin is increased or enhanced in an ES cell. In yet another embodiment, a Ronin activator is any compound, molecule, biological agent or chemical agent that partially or totally stimulates, increases, promotes, increases activation, activates, sensitizes, or up-regulates a protein, a gene, or mRNA stability, expression, function, or activity of Ronin.

In another embodiment, the present invention provides a method of maintaining the self-renewal capacity of an ES cell by stimulating, enhancing, or activating the expression of Ronin or a Ronin activator by an ES cell. In one embodiment, Ronin or a Ronin activator may be constitutively expressed by an ES cell. In another embodiment, Ronin or a Ronin activator may be expressed conditionally by an ES cell by controlling an inducible promoter that governs expression of Ronin or a Ronin activator. As described elsewhere herein, when an inducing agent activates an inducible promoter, expression of Ronin or a Ronin activator is increased.

In another embodiment, the present invention provides a method of preventing the differentiation of an ES cell by stimulating, enhancing, or activating the expression of Ronin or a Ronin activator by an ES cell. In one embodiment, Ronin or a Ronin activator may be constitutively expressed by an ES cell. In another embodiment, Ronin or a Ronin activator may be expressed conditionally by an ES cell by controlling an inducible promoter that governs expression of Ronin or a Ronin activator. As described elsewhere herein, when an inducing agent activates an inducible promoter, expression of Ronin or a Ronin activator is increased.

In yet another embodiment, the method of the invention includes reversibly preventing the differentiation of an ES cell by stimulating, enhancing, or activating the expression of Ronin or a Ronin activator by an ES cell. In one embodiment, Ronin or a Ronin activator may be constitutively expressed by an ES cell. In another embodiment, Ronin or a Ronin activator may be expressed conditionally by an ES cell by controlling an inducible promoter that governs expression of Ronin or a Ronin activator. As described elsewhere herein, when an inducing agent activates an inducible promoter, expression of Ronin or a Ronin activator is increased and the ES cell does no differentiate. When the inducing agent is removed, Ronin or a Ronin activator expression is reduced and the ES cell differentiates.

### Methods for Expressing a Ronin Activator

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., MOLECULAR CLONING: A LABORATORY MANUAL, volumes 1-3 (3^{rd} ed., Cold Spring Harbor Press, NY 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape.

Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Certain post-translational modifications are the result of the action of recombinant host cells on the expressed polypeptide. Glutaminyl and aspariginyl residues are frequently post-translationally deamidated to the corresponding glutamyl and aspartyl residues. Alternatively, these residues are deamidated under mildly acidic conditions. Either form of these residues falls within the scope of this invention.

Other post-translational modifications include hydroxylation of proline and lysine, phosphorylation of hydroxyl groups of seryl, threonyl or tyrosyl residues, methylation of the α-amino groups of lysine, arginine, and histidine side chains (T E Creighton (1983) Proteins: Structure and Molecular Properties, W.H. Freeman & Co., San Francisco, pp. 79-86).

Regardless of the method used to introduce exogenous nucleic acids into a host cell or otherwise expose a cell to the nucleic acid, in order to confirm the presence of the recombinant DNA sequence in the host cell, a variety of assays may be performed. Such assays include, for example, “molecular biological” assays well known to those of skill in the art, such as Southern and Northern blotting, reverse transcription polymerase chain reaction (RT-PCR) and PCR; “biochemical” assays, such as detecting the presence or absence of a particular peptide, e.g., by immunological means (ELISAs and Western blots).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Culture, Differentiation, Alkaline Phosphatase Staining and Transfection of Cells

Mouse embryonic fibroblasts (MEFs) and HEK293 cells were cultured in Dulbecco's Modified Eagle's Medium (DMEM: Gibco Invitrogen Carlsbad, Calif.) supplemented with 10% fetal bovine serum (FBS; Gibco), L-Glutamine (Gibco), 100 nM non-essential amino-acids (Gibco) and μ100 M beta-mercaptoethanol (Fluka, Milwaukie, Wis.). Mouse ES cells (R1 cells and derivatives), iPS cells and iSCs were cultured in DMEM+GlutaMax I (Invitrogen, Carlsbad, Calif.) supplemented as described for the MEF medium plus 1000 μml. Lif (Millipore Billerica, Mass.) with genetecin (Invitrogen) added at a concentration of 200 μg/ml where relevant. Embryonic stem (ES) cells were co-cultured with irradiated MEFs (or in 0.1% gelatin-coated dishes) in Knock-Out DMEM (Gibco) containing the same supplements as MEF medium, plus 1000 U/ml LIF (Chemicon, Billerica, Mass.).

To differentiate ES cells, we cultured them for 1 day without genetecin and then plated 1×105 cells per 10 cm2 in medium without Lif, followed by an additional 4 days of culture under those conditions. The medium was changed after 2 and 3 days. Afterwards, the cells were washed with PBS, fixed with 2% paraformaldehyde for 30 minutes at room temperature and stained for alkaline phosphatase activity according to the manufacturer's standard protocol (Alkaline Phosphatase Substrate Kit III; Vector Laboratories). Undifferentiated, partially differentiated and totally differentiated colonies were counted (100 colonies per well) in triplicate.

For monolayer differentiation studies using the mpRonin-lacZ reporter ES cell line (clone C) and wild-type (wt) R1 ES cells, cells were plated onto gelatin-coated culture dishes at 6000 cells/cm² in ES cell medium without LIF and cultured for 3 days. Medium was replaced daily. Embryoid bodies (EB) were formed in hanging drops (20 μl mES medium) seeded with 600 cells and cultured for 3 days without LIF. Differentiation of R1, EF1α-Ronin and EF1α-ΔRonin R1ES cells, detected by alkaline phosphatase staining, was induced by plating 1000 cells/cm² and culturing in the absence of LIF for 4 days. For alkaline phosphatase staining, cells were washed in phosphate-buffered saline (PBS), fixed for 30 minutes in 2% paraformaldehyde at room temperature (RT), washed once in PBS and stained in the dark using the AlkPhosIII Kit (Vector Laboratories), as described by the manufacturer. Plasmids were transfected using Lipofectamine-2000 (Invitrogen) following the manufacturer's standard protocol, unless indicated otherwise.

### SELEX

The coding region of mouse Ronin was introduced into pET101/D/lacZ (Invitrogen, Carlsbad, Calif.) to create a mRonin-His/V5 fusion construct. Fusion protein expression was induced in the BL21Star strain of E. coli by treating with 1 mM IPTG for 5 hours. His-tagged Ronin was purified using Ni-NTA Fast Start (Qiagen, Valencia, Calif.) columns according to the manufacturer's protocol and concentrated using YM-10 centrifugal filters (Millipore, Bellerica, Mass.). Briefly, the randomized SELEX template, 5′-TGG GCA CTA TTT ATA TCA AC-N₂₅-AAT GTC GTT GGT GGC CC-3′ (SEQ ID NO. 1; where N₂₅ is a 25-base sequence of randomly inserted nucleotides), was synthesized and amplified with the primers, (+) 5′-CCC GAC ACC CGC GGA TGG GCA CTA TTT ATA TCA AC-3′ (SEQ ID NO. 2) and (−) 5-CGC GGA TCC TAA TAC GAC TCA CTA TAG GGG CCA CCA AC GAC ATT-3′ (SEQ ID NO. 3) in a PCR reaction using 10 μM of each oligonucleotide. PCR cycling conditions used were: 94° C. for 5 minutes, 30 cycles of 94° C. (1 minute), 50° C. (1 minute) and 72° C. (1 minute), with a final 10 minute extension at 72° C. NTA agarose bead-conjugated Ronin was prepared by adding 1 μl (550 ng) recombinant Ronin-His protein to 3 μl washed Ni-NTA agarose beads in 100 μl NT2 buffer (20 mM Tris, pH 7.5; 100 mM NaCl; 0.05% NP40). After a 30 minute incubation at 4° C. beads were washed twice and resuspended in 100 μl binding buffer (20 mM Tris, pH 7.5; 100 mM NaCl; 0.05% NP40; 0.5 mM EDTA; 100 μg/ml BSA; 50 μg/ml Poly dI-dC). One hundred microliters (3 μg) of purified PCR product (PCR Purification Kit; Qiagen) was added to Ronin-conjugated beads and incubated for 5 minutes at RT, followed by 5 washes with NT2. Beads were resuspended in 100 μl H₂O and Ronin-bound DNA was purified by phenol/chloroform extraction and ethanol precipitation, then resuspended in 10 μl H₂O. Two microliters of DNA isolated from the first round of Ronin binding were used in a second round of PCR amplification, purification and Ronin-binding using the same conditions. Seven rounds of PCR amplification were used in the Selex procedure, generating a population of PCR products enriched for specific Ronin binding. These products were then ligated into the pGEM-TEasy vector (Promega, Madison, Wis.). One hundred and four randomly selected clones were sequenced and analyzed using the Multiple EM for Motif Elicitation software (meme.sdsc.edu).

### Targeted Deletion of the Mouse Ronin Gene

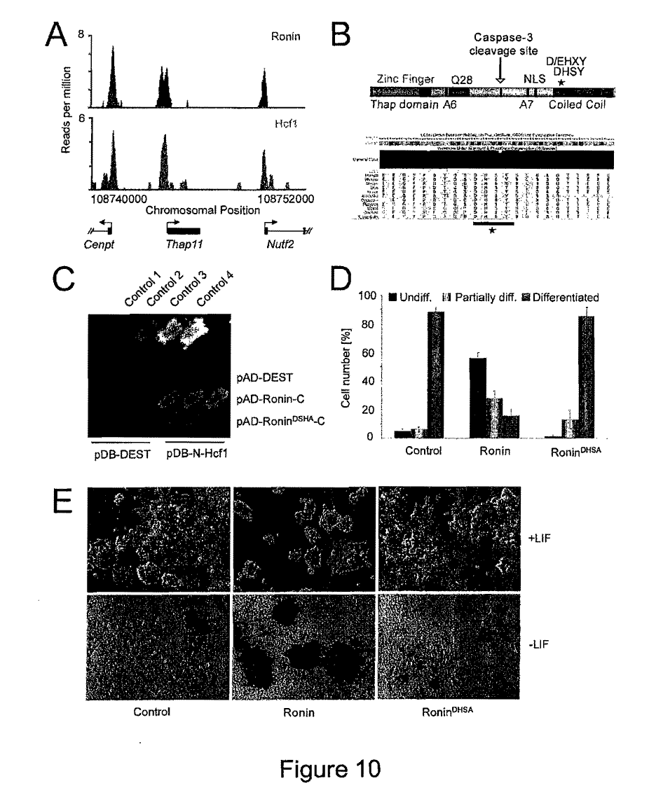

A targeting vector specific for the mouse Ronin allele was created in a four-step cloning procedure using the pfrt-loxP plasmid as a backbone (a gift from Dr. James Martin, Texas A&M Institute of Biotechnology). This vector contains loxP sites separated by a multiple cloning site, a PGK-neomycin-resistance cassette (NeoR) flanked by Flp recombinase recognition sites, and a downstream thymidine kinase gene for negative selection. The entire Ronin mRNA coding region was inserted between the loxP sites, which were placed in regions of low homology to create an inducible null genotype (FIG. 10A). After linearization with AscI, the Ronin targeting vector was introduced into R1 ES cells by electroporation followed by selection in the presence of G418 and ganciclovir; 960 G418- and ganciclovir-resistant colonies were isolated. After screening 56 individual ES cell colonies by PCR analysis, four positive clones were identified, designated Ronin^{+/flox}; three of these were subsequently microinjected into blastocyst-stage embryos and implanted into pseudopregnant female recipients to generate chimeric mice (Mouse Embryo Manipulation Services at Baylor College of Medicine). After confirming the genotypes of the resulting mice by Southern blotting and PCR analysis of genomic DNA (FIG. 10B), the mice were crossed with Zp3-Cre transgenic mice (The Jackson Laboratory), Female mice carrying the deleted Ronin allele and Zp3-Cre transgene were then crossed with B6 wild-type males to obtain Ronin^{+/-} (heterozygous) offspring.

### Generation of EF1α-Ronin ES Cells

EF1α-Ronin mouse ES cells, in which constitutive, ectopic expression of FLAG-tagged human Ronin could be eliminated by a Cre recombination event, were generated by introducing FLAG tag and loxP sites and the desired restriction sites using PCR, as described in Supplementary Experimental Procedures. The resulting PCR product was ligated into BglII and XbaI sites of the pEF1-luciferase-IRES-Neo vector (a generous gift from David Spencer, BCM), replacing the luciferase gene with the Ronin coding sequence to generate the pEFI-hRonin-Flag-loxP vector. Twenty micrograms of circular vector were linearized with the restriction enzyme. NdeI, and electroporated into 10×10⁶ R1 mouse ES cells, which were then grown on a layer of neomycin-resistant MEF feeder cells. Transfectants were selected over a period of 8 to 10 days using 200 μg/ml Geneticin (Invitrogen), after which individual ES cell colonies were screened for Ronin expression by Western blot analyses using an antibody against the FLAG epitope (Sigma, St. Louis, Mo.). Under denaturing conditions, Ronin migrated as a 50 kD protein. A control cell line expressing the pEF1/His/C vector (Invitrogen) was produced in a similar manner using NruI-linearized vector.

Generation of EF1α-ΔRonin ES cells

To eliminate ectopic Ronin expression and generate EF1α-ΔRonin ES cells, EF1α-Ronin ES cells were plated at a density of 3600 cells/10-cm dish and cultured without LIF. After 4 days, cells grown on 10-cm dishes were re-plated at the same density in 15-cm dishes and selected in medium without LIF for 4 more days. Colonies were then selected and expanded on MEF feeder cells in medium supplemented with LIF. After expansion, 5×10⁶ cells were plated on MEFs in a 10-cm dish and transfected 4 hours later with a 1:4 mixture of pCMV-GFP (Stratagene, La Jolla, Calif.) and pSalk-Cre. After culturing for 18 hours, 5×10⁶ GFP positive cells were sorted by fluorescent activated cell sorting (FACS; BD Biosciences FACSaria) and 2,500 green fluorescent cells/cm² were plated on MEFs in a 10-cm dish. After passaging twice, the GFP signal had faded completely, indicating that Cre activity was absent. Passaged cells were then plated at 1,000 cells/cm² and cultured for 9 days to allow colonies to form. Selected colonies were expanded and genotyped for successful elimination of Ronin by Cre-mediated recombination. Thirty clones were genotyped using the oligos MAD221 (5′-CCG GCC TTA TTC CAA GCG GC-3′; SEQ ID NO. 4) and MAD224 (5′-CTG ACT GCT GTC TAC AGT GGC CTG-3′; SEQ ID NO. 5). A second PCR, using oligos MAD221 and MAD222 (5′-AGT CAG GCT CCG GGA TCC GTA CAG-3′; SEQ ID NO. 6), was performed to exclude the presence of expanded mixed cultures containing non-revertant EF1α-Ronin ES cells. After culturing in the absence of LIF for 4 days, as described, six of these clones were shown to differentiate in a manner similar to wt R1 cells, three of which generated chimeric mice following injection into blastocysts, confirming pluripotency. Chimeric mice were identified at 3 weeks of age on the basis of coat color.

siRNA Knockdown Experiments

To induce differentiation by knocking down oct3/4, R1 cells (1×10⁵/well) or EF1α-Ronin ES cells (2×10⁵/well), plated in 6-well plates (10 cm²/well), were transfected with Smart Pool siRNA oct3/4 (Dharmacon, M-046256-00-0005; Chicago, Ill.) using 5 μl of Lipofectamine-2000 and following the siRNA transfection protocol for D3 cells (Invitrogen, Carlsbad, Calif.). Briefly, Lipofectamine-2000 and siRNA were diluted in 250 μl OptiMEM, incubated for 15 minutes, mixed, incubated for an additional 15 minutes, and then added to cells. GFP duplex siRNA (Dharmacon, Chicago, Ill.) served as a negative control in a parallel experiment. Cell morphology was assessed by alkaline phosphatase staining after 3 days.

### Teratoma Formation

To induce teratoma formation, 1×10⁶ cells from each cell population were injected into the quadriceps muscle of the hind legs of immunocompromised Fox Chase SCID beige mice (The Jackson Laboratories, Bar Harbor, Me.). Teratomas were dissected after 16 days. A small piece of each tumor was used in PCR analyses to confirm genotypes. Half of the remaining tissue was fixed in 0.4% paraformaldehyde and embedded in optimal cutting temperature (OCT) medium (Sakura Finetek Inc) after incubating overnight in 30% sucrose; the remaining half was fixed in 10% Formalin overnight, transferred to 70% ethanol and paraffin embedded. Paraffin-embedded sections were analyzed by hematoxylin and eosin (H&E) staining. OCT-embedded tissue was used for X-Gal staining, as described.

### 5-Fluorouridine Staining of Newly Transcribed RNA

A172loxP or A172LP-Ronin-FLAG cells (1.5×10⁵ cells/well) were plated on an MEF feeder layer in 2-chamber slides. After 8 hours, Ronin expression was induced with doxycycline (1 μg/ml), and 12 hours later nascent RNA was labeled by incubation with 100 μM 5-fluorouridine (5-FU, Sigma, F5130) for 1 hour, as described by Boisvert et al. (2000). 5-FU was detected with an anti-BrdU primary antibody (Sigma, 1: 500) and goat anti-mouse Alexa Fluor 488 secondary antibody (Molecular Probes, 1: 1000). Ronin was detected with the Ronin antiserum, 04275 (1: 2000), and the secondary antibody, AlexaFluor 594 (Molecular Probes, 1: 1000). Cells were mounted in Vectashield Mounting medium with DAPI (Vector Laboratories, Bulinghame, Calif.) and examined by fluorescence microscopy using a Zeiss 63×/1.40 objective. Images were deconvoluted using the Resolve3D software.

### Directional Yeast Two-Hybrid Screen for Potential Ronin-Interacting Proteins

A total of 32 cDNA sequences corresponding to proteins identified by mass spectrometry were amplified by PCR from corresponding human cDNA clones (Open Biosystems). Sequence-specific oligonucleotides with attB overhangs were used, generating PCR products that were subsequently cloned into pDONR221 (Invitrogen) using the Gateway technology, according to the manufacturer's protocol (Invitrogen). Ronin, an N-terminal fragment corresponding to amino acids (aa) 1-103, and a C-terminal fragment corresponding to aa 132-315 were amplified from pEF1-hRonin-FLAG and introduced into pDONR221 using the same strategy. The specific primers used were: 1) Ronin, MAD 110 (5′-GGG GAC AAC TTT GTA CAA AAA AGT TGG CAT GCC TGG CTT TAC GTG CTG CG-3′; SEQ ID NO. 7) and MAD111 (5′-GGG GAC AAC TTT GTA CAA GAA AGT TGG TCA CAT TCC GTG CTT CTT GCG G-3′; SEQ ID NO. 8); 2) aa 1-103, MAD110 and MAD207 (5′-GGG GAC AAC TTT GTAC AAGA AAGT TGGT TAC CTG CGG CGG GCG GCC GCG GCC CCA GC-3′; SEQ ID NO. 9); aa132-315, MAD208 (5′-GGG ACA ACT TTG TAC AAA AAA GTT GGC TCC TCA CCC TCT GCC TCC ACT GCC-3′; SEQ ID NO. 10) and MAD111. The HCF-1 cDNA clone represents aa 1-429 and the Sin3a sequence corresponds to aa 1-123 of the mature protein; all others code for full-length proteins. cDNAs were subsequently shuttled into the yeast two-hybrid destination vectors, pDEST-DB and pDEST-AD. The resulting vectors expressed fusion proteins with an N-terminal activation domain or a DNA binding domain (reflecting the corresponding functional requirements of the directional screen) and screened using the Proquest yeast two-hybrid system (Invitrogen) following the manufacturer's protocol. Briefly, vectors expressing Ronin or the truncated versions of Ronin were co-transfected into the yeast strain, MAV 103, with the corresponding vectors expressing the potential Ronin-interacting protein. Transfected yeast were then selected on -Trp-Leu plates and evaluated for growth phenotype, as described by the manufacturer (Invitrogen). All potential interacting proteins were tested as activation-domain fusions and DNA-binding-domain fusions to overcome functional repression associated with either fusion context.

Immunoprecipitation with GST-Bound Agarose Beads

cDNAs for Ronin, and N- and C-terminal Ronin deletion mutants (see above), were shuttled into the pMyc-DEST vector and the pGST-DEST vector (Invitrogen) using the gateway technology to generate fusion proteins carrying an N-terminal Myc tag or GST domain, respectively. Co-transfection of HEK293 cells, GST-immunoprecipitation and detection of precipitated myc-tagged proteins were performed. Briefly, HEK293 cells were plated at 1×10⁶ cells/well in a six well plate and were co-transfected after 24 hours with expression vectors for two potential interaction partners (one in pMyc vector, the other in pGST vector; 0.8 μg each). Forty-eight hours later, cells were lysed in lysis buffer (20 mM Tris pH 8, 180 mM NaCl, 1 mM EDTA, 0.5% NP-40, Protease Inhibitor Complete (Roche, Nutley, N.J.)) and cell lysates were immunoprecipitated using glutathione sepharose 4B (GE Healthcare, Piscataway, N.J.). After washing, beads were resuspended in 12.5 μl SDS loading buffer, heated at 70° C. for 10 minutes, separated on 4-15% Tris-HCl gradient gels (Biorad) and analyzed by Western blotting using standard procedures. The following antibodies (dilutions) were used: rabbit anti-glutathion-5-transferase (Sigma, 1: 2000), mouse anti-c-myc (Sigma, clone 9E10, 1: 2000), HRP-conjugated sheep anti-mouse IgG (GE Healthcare, 1: 4000) and HRP-conjugated donkey anti-rabbit IgG (GE Healthcare, 1: 10000). Signals were developed using the ECL system (GE Healthcare) following the manufacturer's protocol. An aliquot of the cell lysate (input) was tested to verify expression of the corresponding GST and myc proteins.

### Generation of the Ronin-Inducible Mouse ES Cell Line, A172LP-Ronin-Flag

To generate an inducible cell line, a FLAG tag was first added to the 3′ end of Ronin in a PCR reaction using the primers, (+) 5′-GTC GCA GCC ATG CCT GGC TTT ACG-3′ (SEQ ID NO. 11) and (−) 5′-TCA CTT ATC GTC GTC ATC CTT GTA ATC CAT GCC GTG CTT CTT ACG GAT G-3′ (SEQ ID NO. 12). The purified product was cloned into pGEM-Teasy (Promega), digested with EcoRI and ligated into the corresponding EcoRI site of p2Lox-EGFP, replacing EGFP with mRonin. Twenty micrograms of the resulting vector, p2Lox-Ronin-CFlag(C15), and 20 mg of pSalkCre were simultaneously electroporated into 1.5×106 A172LoxP cells, and plated onto neomycin-resistant MEFs. Individual clones were selected and expanded in the presence of 400 mg/ml geneticin. Ronin expression was verified by Western blotting and immunofluorescence using an anti-FLAG antibody after induction with 100 ng/ml to 1 mg/ml doxycycline for 6 to 80 hours.

### Gel Mobility Shift Experiments

Gel mobility shift experiments were performed using the LightShift Chemiluminescent EMSA kit, according to the manufacturer's instructions (Pierce, Rockford, Ill.). Nuclear extracts were prepared from A 172LP-Ronin-Flag cells after induction with 1 mg/ml doxycycline for 12 hours. First, microscopically intact nuclei were obtained as described by Remboutsika et al., 1999, J. Chem. Sci. 112:1671-1683. Intact nuclei were then lysed on ice for 20 minutes in NIB buffer (15 mM Tris pH 7.2; 60 mM KCl; 15 mM MgCl2; 15 mM NaCl; 1 mM CaCl2; 1 mM PMSF; 5 mM sodium orthovanadate; 5 mM sodium fluoride; 500 mM dithiothreitol; protease inhibitor cocktail) supplemented with 0.6% NP40. Lysates were centrifuged at 2000×g for 5 minutes at 4° C. and the supernatant, containing the nuclear contents, was stored in 25% glycerol at −80° C. until ready for use. The biotin-labeled 3× sequence, 5-bio-ATC AAC TGT ATA CAA GCA GCT AGG ACA GCA CCC TAA TGT C-3′ (SEQ ID NO. 13), and the unlabeled 3× sequence, 5′-TGT ATT ACA AGC TAG GAC AGC ACC T-3′ (SEQ ID NO. 14), were used as probes. Sense and anti-sense strands were synthesized and hybridized in equimolar amounts for use as a double-stranded template. Two microliters of 20 nM biotin-labeled 3× double-stranded DNA was mixed with 4 ml (3 g) nuclear extract in a total volume of 20 ml. To block binding of Ronin to the template, 1 mg/ml anti-FLAG antibody (M2, Stratagene) was preincubated with 10 mM unlabeled double-stranded 3× or single-stranded 3× sequence and nuclear extract. Normal mouse IgG (Santa Cruz) was used in the control reaction. Oligos used in competition experiment: Oligo A: 5′-GGA CAG CAC CCT-3′ (SEQ ID NO. 15), Oligo B: 5′-CAA GCT AGG ACA G-3′ (SEQ ID NO. 16) and Oligo C: 5′-TGT ATT ACA AGC TAG GAC AGC ACC CT-3′ (SEQ ID NO. 17). Generating probes for Northern and Southern blot hybridization Ronin Probe 1 was generated by PCR using the GoTaq Green mastermix (Promega) and the primers, (+) 5′-GGA TCC GTG GTT CCC GTG-3′ (SEQ ID NO. 18) and (−) 5′-CCA TGG CAA GCA GAC GAT C-3′ (SEQ ID NO. 19) under the following cycling conditions: 98° C. for 15 seconds, 40 cycles of 95° C. for 10 seconds, 52° C. for 30 seconds and 72° C. for 30 seconds, followed by a final extension at 72° C. for 7 minutes. Ronin probe 2 was amplified using the primers, (+) 5′-GTG GGT CGC TAA ACC TGA GAG-3′ (SEQ ID NO. 20) and (−) 5′-GTT CAA TGA GTT AGC TGT GTC-3′ (SEQ ID NO. 21) under the same conditions as used for Ronin probe 1. The GAPDH probe was generated using the primers, (+) 5′-ACC ACA GTC CAT GCC ATC AC-3′ (SEQ ID NO. 22) and (−) 5′-TCC ACC ACC CTG TTG CTG TA-3′ (SEQ ID NO. 23) under the same PCR cycling conditions, except that an annealing temperature of 55° C. was used. Five nanograms of mouse genomic DNA was used as template for all probes. The PCR products were purified by agarose gel extraction (Qiagen) and labeled with ³²P using the Rediprime H Random Prime labeling system following the manufacturer's protocol (Amersham/GE Healthcare). Seventy-five nanograms of the labeled probes were used in subsequent Northern or Southern blot hybridizations.

### Northern Blot Analysis

Total RNA from R1 ES cells and adult mouse tissues was extracted using the UltraSpec RNA reagent (Biotex, Houston, Tex.). For Northern blot analysis, total RNA (10 mg) was separated on a 1% agarose gel in 1×MOPS buffer in the presence of 8% formaldehyde, transferred to a charged nylon membrane (Millipore) and crosslinked with UV light. The membrane was hybridized in Express Hyb Hybridization solution (Clontech, Mountain View, Calif.) at 68° C. overnight using ³²P-labeled Ronin probe 1. The membranes were washed three times for 1 hour with wash buffer 1 (2×SSC, 0.1% SDS), two times for 1 hour with wash buffer 2 (0.1×SSC, 0.1% SDS) followed by an overnight wash in wash buffer 2. All washes were carried out at 68° C. Membranes were then exposed to X-Ray film for 6 hours to 2 days at −80° C.

### Southern Blot Analysis

Genomic DNA was isolated from tail biopsies following digestion with proteinase K using phenol-chloroform extraction. DNA (10 mg) was incubated with the indicated restriction enzyme overnight and then separated on 0.8% agarose gels. DNA was denatured in situ and then transferred to a nylon membrane (Millipore). After crosslinking DNA to the membrane, the membrane was hybridized with the indicated 32P-labeled cDNA probe (see above). Probe labeling, hybridization and washes were done as described for the Northern blot hybridization (see above), but hybridization and washes were performed at 55° C. The additional overnight wash was only included when Ronin probe 1 was used.

### Production of Ronin Antiserum

Ronin antiserum was made by GeneMed Synthesis, Inc. Three peptides, VPG CYN NSH RDK ALH (SEQ ID NO. 24), TGS DHS YSL SSG TTE (SEQ ID NO. 25) and LME VKM KEM KGS IRH (SEQ ID NO. 26), corresponding to sequences conserved between mouse and human were synthesized; each was KLH-conjugated and contained an N-terminal cysteine. Two separate rabbits were immunized with a mixture of all three peptides on day 1, 20, 40 and 60. Anti-sera were collected from each rabbit ten days after the fourth immunization. Rabbit anti-Ronin lot G4275 and the corresponding pre-immune serum were used in all experiments.

### Immunofluorescence of Mouse and Human ES Cells, Ovarian Tissue and Zygotes

Ovaries from a superovulated C57B/6 wild-type female were fixed in 4% paraformaldehyde and embedded in paraffin. Sections (5 mm) were stained with a 1:50 dilution of the anti-Ronin or pre-immune serum and detected with the secondary Alexa Fluor 488 anti-rabbit antibody (Molecular Probes, Carlsbad, Calif.; 1:2000 dilution). Sections were mounted in Vectashield mounting medium with DAPI (Vector Laboratories) and immunofluorescence was visualized using a deconvolution microscope. For immunostaining of Ronin in mouse and human ES cells, H9 or R1 cells were grown on glass coverslips and fixed for 30 minutes at 4 C in 2% paraformaldehyde dissolved in PBS. After three washes in PBS, the cells were permeabilized for 30 minutes at room temperature with 0.3% Triton-X-100 in PBS and blocked over night at 4 C in Buffer G.

The cells were washed one hour in PBS and then incubated for one hour at room temperature with the Ronin immune serum (described), and pre-immune serum as control, diluted 1:5000 in Buffer G. After three washes for 15 minutes in PBS/0.1% Triton-X-100, secondary antibody in Buffer G (1:2000), was added for one hour at room temperature. After three washes for 15 minutes in PBS/0.1% Triton-X-100 and two washes for 15 minutes in PBS, the cells were fixed in 0.2% formaldehyde in PBS for 30 minutes at 4° C. and washed twice in PBS. Slides were mounted in Vectashield with DAPI (Vector Laboratories). For immunostaining of early embryos, they were isolated from wild type 129×1/SVJ mice following the protocol described by Nagy et al (Nagy, 2003, Cold Spring harbor Laboratory Press, ISBN 0-87969-591-9). E2.5 or E3.5 embryos were washed twice with 1×BD perm/wash buffer (BD Pharmingen, San Jose, Calif.) and then fixed with Cytofix/Cytoperm buffer (BD Pharmingen) at 4° C. for 1 hour. Embryos were washed twice and then blocked in 1×BD perm/wash buffer for an additional hour at room temperature. The embryos were then incubated at 4° C. overnight shaking gently in a 1:1000 dilution in 1×BD perm/wash buffer of anti-Ronin G4275 or the pre-immune serum control. Embryos were washed by moving them through small drops of 1×BD perm/wash buffer at room temperature and then incubated with the secondary antibody (Alexa Fluor 488 goat anti-rabbit IgG, Invitrogen) in a 1:2000 dilution in 1×BD perm/wash buffer at 4° C. overnight shaking gently. Embryos were washed as before and mounted in Vectashield with DAPI (Vector Laboratories Inc.). Signals were visualized with a fluorescence microscope (Zeiss Axioplan 2), images were taken with the MetaVue (Version 6.1r1) software. E3.5 blastocysts were plated on 0.1% gelatinized 24-well culture dishes and grown for 3 days to detect Ronin expression in ICM outgrowth following the same staining protocol with a 1:5000 dilution of the primary antibodies.
Generation of mpRonin-lacZ Reporter Mouse Line

An mpRonin-lacZ reporter construct was generated by PCR amplification of a 3.3 kb genomic region of the mouse Ronin gene that included a region upstream of the translation initiating ATG codon and the first 15 base pairs within the annotated coding region. PCR was performed using native Pfu Polymerase (Stratagene) in the presence of 4% DMSO and the primers (+) 5′-ACA AAG CTT AGT CTC GCG ATG CTG CCA C-3′ (SEQ ID NO. 27) and (−) 5′-ACA GCT AGC CGT AAA GCC AGG CAT GGC TG-3′ (SEQ ID NO. 28). The PCR program consisted of an initial denaturation at 98° C. for 1 minute, 35 cycles of 96° C. for 15 seconds, 55° C. for 1 minute and 68° C. for 8 minutes, and a final extension at 68° C. for 10 minutes. The PCR product was ligated into the HindIII and NheI sites of the pEF1/His/LacZ expression vector (Invitrogen), exchanging the pEF1a-His region for the Ronin promoter region. The resulting mpRonin-lacZ vector was linearized with Hind III and injected into the pronucleus of wt C57B/6 oocytes (Mouse Embryo Manipulation Services MEMS) Baylor College of Medicine). The resulting mice were genotyped using GoTaq DNA Polymerase (Promega) and the primers, lacZ(+) 5′-CTT AGG ACG AGC TTC ATC TG-3′ (SEQ ID NO. 29) and lacZ(−) 5′-GAC GGG ATC AAC TCC AAG CTG-3′ (SEQ ID NO. 30) with the following cycling conditions: 95° C. for 3 minutes, 40 cycles of 94° C. for 30 seconds, 55° C. for 30 seconds and 72° C. for 30 seconds, followed by a final extension at 72° C. for 7 minutes. Three of seven PCR-positive transgenic mouse lines (8, 9 and, 15) were analyzed for ovarian lacZ expression; post-implantation studies were done using line #8. For pre-implantation studies, a transgenic male from line #8 was crossed with 3-week-old superovulated wt C57B/6 females. Line #15 was used for X-Gal staining of adult mouse tissues.

### X-Gal Staining of Adult Tissues, Tissue Sections, Cultured Cells and EBs

Adult mice were perfused with 4% Paraformaldehyde, tissues were dissected and postfixed for 1 hour at 4° C., washed three times in X-Gal rinse buffer (PBS containing 2 mM MgCl₂, 0.25% sodium deoxycholate and 0.2% triton X-100) at RT for 30 minutes and then stained in X-Gal staining solution (40 mg/mL X-gal in 2 mM MgCl₂, 5 mM K₄Fe(CN)6×3H₂O and 5 mM K₃Fe(CN)₆) overnight at 37° C. Specimens were then post-fixed in 2% paraformaldehyde for 1 hour and stored in 70% ethanol.

Cultured cells and EBs were fixed for 5 minutes at room temperature in 2% formaldehyde/0.2% glutaraldehyde, washed four times (10 minutes each) in wash buffer, submerged in X-Gal staining solution for 2 to 16 hours, post-fixed in 2% paraformaldehyde and stored in PBS at 4° C.

For X-Gal staining of sections from OCT embedded tissue, 10 mm sections were post-fixed for 15 minutes in 4% paraformaldehyde, rinsed in PBS and then washed (10 minutes) in PBS containing 2 mM MgCl₂, followed by two 10-minute washes in rinse buffer. All steps were preformed on ice. Sections were stained overnight at 37° C. in X-Gal staining solution, washed twice (5 minutes each) in PBS, rinsed in H2O, counterstained with Nuclearfast Red, dehydrated and mounted in Permount (Fisher Scientific, Pittsburgh, Pa.).

### Histology and Embryo Collection

Preimplantation embryos were isolated from 3.5- to 4.5-week-old mpRonin-lacZ or Ronin^{+/-} female mice, superovulated using pregnant mare serum (5 units) followed 47 hours later by human coriogonadotropic hormone. These females were then mated with wt or Ronin^{+/-} males and examined the following morning for vaginal plugs. Embryos were flushed from the uterus 3 days after identifying vaginal plugs or removed from the oviduct the same day. After removal, embryos were either fixed or cultured. For histology, the uterus was fixed, paraffin-embedded and H&E-stained after sectioning (according to Behringer et. al., “Mouse phenotypes”). DNA for genotyping was isolated from embryos dissected from the deciduas and cleared of maternal tissue.

### Embryo Culture and ES Cell Derivation

For ICM outgrowth experiments, individual blastocysts isolated from Ronin^{+/-} crosses were transferred to a well of a gelatin-coated 24-well culture dish in ES cell medium containing 20% FCS and cultured for 4 days. Cultures were genotyped using PCR. To derive ES cells, blastocysts were cultured for 3 days on gelatin, as above, and then expanded on an MEF feeder layer. Newly derived ES cell lines were genotyped using PCR and Southern blotting.

### Generation of the Ronin Targeting Vector

A targeting vector specific for the mouse Ronin allele was created using the plasmid pfrt-loxP as a backbone in a four step cloning procedure. Using genomic DNA isolated from R1 ES cell as template, four PCR fragments were generated with Pfu Polymerase (Stratagene), sequence-verified and ligated into pfrt-loxP. A 3′ short homologous arm (1.5 kb), amplified using the primers, (+) 5′-ACA CTC GAG TAGA TAG GTA TTG GCC TAT TTG AAA GAA C-3′ (SEQ ID NO. 31) and (−) 5′-ACA CCT AGGT GGC ACA TAC CTT TAA TCC CAG CAC-3′ (SEQ ID NO. 32), was first ligated into the XhoI and AvrII restriction sites. The Ronin mRNA region (2 kb) was split into two fragments and sequentially ligated (3′ end of the gene followed by the 5′ end) into the NotI site in pfrt-loxP using the corresponding NotI site within the Ronin gene. The 3′ end of the gene was amplified using the primers (+) 5′-AGG GC GGC CGC AAG ACC TAC ACG GTG-3′ (SEQ ID NO. 33) and (−) 5′-ACA CGG CCG ATC CCC ACA TTT CAA GGA CA CTT AGC T-3′ (SEQ ID NO. 34) and ligated using the EagI site (introduced into the (−) primer) to preserve NotI as a unique site. The 5′ end of the gene was amplified using the primers (+) 5′-ACA GC GGC CGCT ACC TTT CGC TTA GGA CGAG CTT CATC-3′ (SEQ ID NO. 35) and (−) 5′-CTT GCG GCC GCC CTG AAA GTG GAC GCT GCA G-3′ (SEQ ID NO. 36). Finally, a 5′ long homologous arm (8 kb) was amplified using the primers (+) 5′-ACA GGC GCG CCA CGT CTA CGC TAA CTC TGG CAC TGG-3′ (SEQ ID NO. 37) and (−) 5′-ACA ACG CGT TGT ATT CGA ATG GAC ACG TTA TGG C-3′ (SEQ ID NO, 38) and ligated into the AscI site. To preserve AscI as a unique site, the 3′ end of the long arm was ligated into the MluI site.

### Generation of Ronin^{flox/flox} mES Cell Line by Gene Targeting

Cells from the original Ronin^{+/flox}-targeted ES cell clones used for generating the conditional Ronin mouse model (see above) were transfected with pFLP-Cre-IRES-GFP and sorted by FACS to isolated GFP-positive cells. To screen for cells in which the Neor cassette had been deleted, individual colonies were selected and expanded on an MEF feeder layer, then tested for sensitivity to geneticin; Neor deletion was confirmed by PCR. The resulting Ronin^{+/flox} Neor cells were electroporated with the Ronin targeting vector (see above) to introduce loxP sites into the second allele, followed by selection with geneticin and ganciclovir, and genotyping. Clone A3 was found to be successfully Ronin^{flox/flox}-targeted and was used for subsequent studies.

Cre-Mediated Excision of loxP-Flanked Ronin in Ronin^{flox/flox} ES Cells

Ronin^{flox/flox} ES cells were plated onto MEFs at a density of 100,000 cells/cm² and co-transfected with a 4:1 mixture of pSalk-Cre or pEF1a-C and pGFP. Twenty hours after transfection, GFP-positive cells were isolated by FACS (with the expectation that GFP-positive cells would also express Cre-recombinase) and plated onto an MEF feeder layer in a 10-cm dish (30,000 cells/dish). After 7 days, 96 individual colonies were selected, expanded on MEFs and genotyped by PCR. After removing selected colonies, the remaining cells were fixed and stained with crystal violet.

Cloning of pMAX-Cre-GFP

To generate the pMAX-Cre-GFP expression vector, the Cre gene was amplified using the primers (+) 5′-ACA GCT AGC GCC ACC ATG TCC AAT TTA CTG ACC GTA CAC-3′ (SEQ ID NO. 39) and (−) 5′-ACA CCC GGG AGA TCG CC ATC TTC CAGC AGGC GCA C-3′ (SEQ ID NO. 40). The PCR product was cut with NheI and AgeI and ligated into the corresponding sites of pMAX-GFP (Amaxa Biosystems, Walkersville, Md.).

Nucleofection of Ronin^{flox/-} and Ronin^{+/flox} ES cells

A 6-cm plate of confluent cells was used for nucleofection, employing the Mouse ES cell Nucleofector Kit and following the manufacturer's standard protocol in combination with electroporation program A-30 (Amaxa). Ronin^{-/flox} ES cells were nucleofected with pMAX-GFP (Amaxa), and Ronin^{+/flox} ES cells were nucleofected with pMAX-Cre-GFP. After nucleofection, cells were plated into one well of a 6-well plate on an MEF feeder layer, and Cre activity was assessed 48 hours later by GFP expression.

### Cre Virus Transduction of MEFs

MEFs were isolated from embryos derived from Ronin^{flox/flox} P mice and split into one well of a 6-well dish at P1 or P2. When cells reached approximately 50% confluence, they were transduced with adenovirus containing CMV-GFP or CMV-Cre-IRES-GFP in the presence of the GeneJammer transfection reagent (Stratagene), according to the method of Fouletier-Dilling et. al., 2005, Hum. Gene Ther. 16:1287-1297 Adenoviral supernatant was obtained from the Vector Core Facility at the Center for Gene and Cell Therapy (Baylor College of Medicine). Four hours after transduction, cells were washed twice with medium and then cultured in fresh medium. Cells were split after reaching 90-100% confluence and genotyped by PCR.

### RT-PCR Analysis

For RT-PCR analysis, R1 cells (1500 cells/cm²) and EF1α-Ronin ES cells (2500 cells/cm²) were cultured in the absence of LIF for 1, 2, 3, 4 and 5 days. RNA was isolated using the RNeasy tissue kit following the manufacturer's standard protocol with on-column DNA digestion (Qiagen). Total RNA (1 mg) was reverse transcribed using the ImPromII Reverse Transcription System (Promega) in the presence of 4.8 mM MgCl₂ and oligo-dT to generate cDNA. The expression levels of the tested genes were assessed in subsequent PCR reactions using 1 l of each cDNA. PCR reactions (50 ml total volume) were performed using the GoTaq Green Mastermix (Promega) and 10 mM of each primer under the following cycle conditions: denaturation for 3 minutes at 94° C., followed by variable numbers of cycles (see below) of 30 seconds at 94° C., 30 seconds at 55° C., 30 seconds at 72° C., and a final extension for 7 min at 72° C. Primers, cycle numbers and product size were as follows:

TABLE 1

| | | | No. | Size |
|---|---|---|---|---|
| Primer name | SEQ ID NO. | Primer Sequence | Cycles | (bp) |
| mRonin (+) | SEQ ID NO. 41 | GCC TCA GAG CTA GAG GCT GCT ACG | 27 | 400 |
| mRonin (−) | SEQ ID NO. 42 | TGG AAG GAG TCA CGA ATT CTG CAG | | |
| Oct4 (+) | SEQ ID NO. 43 | GGC GTT CTCT TTGG AAA GGT GTT C | 27 | 312 |
| Oct4 (−) | SEQ ID NO. 44 | CTC GAA CCA CAT CCT TCT CT | | |
| Nanog (+) | SEQ ID NO. 45 | CCA GTGG AGT ATCC CAG C AT | 40 | 237 |
| Nanog (−) | SEQ ID NO. 46 | GAA GTT ATG GAG CGG AGC AG | | |
| Fgf5 (+) | SEQ ID NO. 47 | ATA GCA GTT TCCA GTG GAG CCC TT | 35 | 241 |
| Fgf5 (−) | SEQ ID NO. 48 | TGG ATC GCG GAC GCA TAGG TAT TA | | |
| Sox1 (+) | SEQ ID NO. 49 | TTAC TTC CCG CCA GCT CTT C | 30 | 373 |
| Sox1 (−) | SEQ ID NO. 50 | TGA TGC ATT TTG GGG GTA TCT CTC | | |
| Afp (+) | SEQ ID NO. 51 | TCG TAT TCC AAC AGGA GG | 35 | 174 |
| Afp (−) | SEQ ID NO. 52 | AGG CTTT TGC TT CAC CAG | | |
| T-Brachyury (+) | SEQ ID NO. 53 | ATG CC AAAG AAA GAA ACG AC | 35 | 835 |
| T-Brachyury (−) | SEQ ID NO. 54 | AGA GGC TGT AGA ACA TGA TT | | |
| b-Actin (+) | SEQ ID NO. 55 | GGC CCA GAGC AAG AGA GGT ATC C | 27 | 460 |
| b-Actin (−) | SEQ ID NO. 56 | ACG CA CGA TTT CCC TCT CAGC | | |
| GAPDH (F) | SEQ ID NO. 57 | ACC ACA GTC CAT GCC ATC AC | | |
| GAPDH (R) | SEQ ID NO. 58 | TCC ACC ACC CTG TTG CTG TA | | |
| tgRonin (+) | SEQ ID NO. 59 | CAG GCT CCG GGA TCC GTA CAG | 30 | 605 |
| tgRonin (−) | SEQ ID NO. 60 | CCG GCC TTA TTC CAA GCG GC | | |

To quantify the expression level of GAPDH and β-actin, we analyzed the RT-PCR signals of three independent PCRs after agarose gel electrophoresis and ethidium bromide staining using the AlphaEaseFC (Fluor Chem, Alpha Innotech, San Leandro, Calif.) Software Version 5.0.1 (Alpha Innotech, San Leandro, Calif.). The expression level is displayed as the relative intensity in % based on the integrated density value. Ronin transgene specific primers: tgRonin(+) 5′-AGT CAG GCT CCG GGA TCC GTA CAG-3′ (SEQ ID NO. 59), (−) 5′-CCG GCC TTA TTC CAA GCG GC-3′ (SEQ ID NO. 60) (30 cycles, 605 bp).

### Immunohistological Staining of Oct-4 in Teratoma Sections

Teratoma sections (10 μm) were immunostained with the oct3/4 H-134 rabbit polyclonal primary antibody (Santa Cruz Biotechnology; 1:200 dilution) and the biotinylated anti-rabbit IgG (H+L) secondary antibody (Vector Laboratories, Burlingame, Calif.; 1:200 dilution). The signal was developed using the Rabbit IgG Vectastain ABC-AP Kit (Vector Laboratories) following the manufacturer's recommendations. Sections were counterstained with hematoxylin and mounted in Vectashield mounting medium (Vector Laboratories).

### Microarray Analysis

D3 mouse ES cells were transfected with pEF1/His/C or pEF1a-hRonin-FLAG in a 6-cm dish. Medium was changed 2 hours before and 4 hours after transfection and cells were harvested 24 hours later. RNA was isolated using the RNeasy Mini Kit (Qiagen) with on-column DNase digestion, as described by the manufacturer. Microarray analysis of RNA was performed by the Microarray Core facility at Baylor College of medicine using the GeneChip Mouse Genome 430 2.0 Array (Affymetrix, Santa Clara, Calif.).

### Methyl ³H Uridine Incorporation Assay

R1 and EF1α-Ronin clones were plated in a 96-well plate at a density of 5000 cells/well, incubated overnight and then stimulated with 1 g/ml retinolic acid for the indicated times. Cells were then pulsed with methyl-3H uridine (Perkin Elmer, NET027, Waltham, Mass.) for 3 hours and harvested (Packard Filtermate harvester). Methyl-³H uridine incorporation was measured using a Packard Topocount-NXT Microplate Scintillation and Luminescence Counter.

Detection of Histone H3K9 Di-Methylation after Induction of Ronin Expression

Ronin expression was induced in A 172LP-mRonin-FLAG cells, plated 24 hours previously, by treating with 1 μg/ml doxycycline for the indicated times. After induction, cells were resuspended in lysis buffer (0.5 M NaCl, 20 mM Trizma, 0.5% Triton X-100, 0.5% NP-40, 1 mM EDTA, 0.25% sodium deoxycholate) supplemented with Protease Inhibitor Cocktail Complete™ (Roche), and sonicated (10 pulses at 30% duty cycle) using a Branson Sonifier 450 (Branson Ultrasonics, Danbury, Conn.). Western blot analysis was performed using the Odyssey system following the manufacturer's protocol. Histone H3K9 di-methylation was detected using the mouse monoclonal H3K9me2 primary antibody (Sigma; 1:2000 dilution) and secondary goat anti-rabbit IgG (H+L) Alexa-Fluor 680 antibody (Molecular Probes; 1:10000 dilution). β-actin (control) was detected using a mouse monoclonal (-actin primary antibody (Santa Cruz; 1:2000) and IR-Dye 800-conjugated goat anti-mouse IgG (H+L) secondary antibody (Rockland, Gilbertsville, Pa.; 1:10000 dilution).

### Immunoprecipitation and Mass Spectrometry

D3 cells, plated 24 hours previously on gelatin in 10-cm dishes, were transfected with pEF1α-Luc-Ires-Neo and pEF1α-hRonin-FLAG-Ires-Neo. Six 10-cm dishes were used for each plasmid and the medium was changed 3 hours before transfection. Sixteen hours after transfection, cells were resuspended in lysis buffer (10 mM Tris pH 8.0, 1 mM EDTA, 150 mM NaCl, 0.5% NP-40, 0.5% Triton X-100, 0.25% sodium dodeoxycholate) containing Protease Inhibitor Complete (Roche)), incubated on ice for 15 minutes and sonicated. The lysate was centrifuged and the supernatant was precleared by incubating with a mixture containing 2 mg normal mouse IgG and 20 ml of washed protein A/G beads (Santa Cruz) for 1.5 hours with continuous shaking. FLAG-tagged Ronin was immunoprecipitated from pre-cleared lysates by incubating with 20 ml of protein A/G beads and 2 mg of anti-FLAG antibody (Clone M2, Sigma) for 2 hours at 4° C. Immunoprecipitated proteins were eluted with 5 ml of FLAG peptide (Sigma) at 4° C. for 1 hour and subjected to mass spectroscopic analysis.

Ronin Complex Purification from Nuclear Extracts by Glycerol Gradient Centrifugation

Nuclear extracts, prepared from 2.5×109 EF1α-Ronin ES cells (25×15-cm dishes) as described for HeLa cells by Dignam et. al. (1997, Nucleic Acid. Res. 11:1475-1489), were used to isolate and purify Ronin complexes, as described for the purification of the HCF-1 complex from HeLa cells by Wysocka et al. (2006, Methods 40:339-343). Briefly, the nuclear extract was further purified using wheat germ agglutinin (WGA; Vector Laboratories) agarose; WGA-bound proteins were eluted with N-acetyl glucosamine (Vector Laboratories) and concentrated using Microcon Y-10 filters (Millipore). Protein complexes were separated on a 25-50% glycerol gradient by ultra-centrifugation for 10 hours at 32,000 rpm. Collected fractions were separated by SDS-PAGE, followed by Western blot analysis using an anti-FLAG primary antibody (M2, Sigma; 1:1000 dilution) and HRP-conjugated goat anti-mouse secondary antibody (GE Healthcare; 1:2000 dilution). The secondary antibody was detected using ECL Plus solution following the manufacturer's recommendations (GE Healthcare).

### Chromatin Immunoprecipitation

A172 dells were plated at 2×105 cells per 15 cm culture dish. 16 hours after plating, differentiation was induced by addition of 1 μM Retinoic acid in the absence of LIF. After 7 days chromatin immunoprecipitation (ChIP) was essentially performed. Briefly, cells were treated with 1% formaldehyde for 10 minutes a room temperature to crosslink DNA with bound protein. The crosslinking reaction was stopped with 0.125 M glycine and the cells were harvested by scraping in PBS. The cells were washed twice with PBS, swelled on ice for 10 minutes in LB1 (5 mM Hepes pH 8, 85 mM KCl, 0.05% NP40) and disrupted by douncing five times with a loose pestle. The nuclei were spun down and resuspended in 50 μl LB2 (50 mM Tris pH 7.5, 10 mM EDTA, 1% SDS). Following incubation on ice for 10 minutes, LB3 (167 mM NaCl, 16.7 mM Tris pH 7.5, 1.2 mM EDTA, 1.1% Triton-X-100, 0.01% SDS) was added to a final volume of 1 ml. Samples were sonicated on ice with five pulses for 10 seconds on maximum setting with a Fisher Scientific Model 100 Sonic Dismembrator. 200 μl of the sonicated lysate were mixed with 300 μl LB3 and samples were precleared with 4 μl rabbit IgG (Santa Cruz) or 6 μl pre-immune serum by rolling for 1 hour at 4° C. 45 μl of unblocked protein A bead slurry (GE Healthcare) were added and samples were incubated for an additional hour. After centrifugation, 6 μl of the corresponding antibody were added to the supernatant and immunoprecipitation was performed over night rolling at 4 C. The antibodies were Histone 3 (Abcam, #1791), Ronin anti-serum (as described), Histone 3 (dimethyl K9) (Active Motif, #39239) and Rabbit IgG (Santa Cruz) as control. 50 μl of Protein A bead slurry that had been blocked with Salmon Sperm DNA were added and samples were incubated for 2 additional hours. The samples were washed twice with Buffer B (0.05% SDS, 1% Triton-X-100, 20 mM Tris pH 7.5, 2 mM EDTA, 150 mM NaCl), once with Buffer D (0.05% SDS, 1% Triton-X-100, 20 mM Tris pH 7.5, 2 mM EDTA, 500 mM NaCl), once with Buffer 3 (250 mM LiCl, 1% NP40, 10 mM Tris pH 7.5, 1 mM EDTA, 1% Sodium Deoxycholate), once with Buffer C (0.1% Triton-X-100, 20 mM Tris pH 7.5, 2 mM EDTA, 150 mM NaCl), and once with TE plus 30 mM NaCl. Protease inhibitor (Roche) was added to LB 1, 2, 3 and all wash buffers. The bead-chromatin complexes were resuspended in 300 μl TE

plus 30 mM NaCl and treated with 500 μg/ml RNase (Sigma) and 500 μg/ml Proteinase K (Sigma) for 30 minutes at 37° C. To reverse the crosslinking of DNA bound protein, the samples were incubated for 20 hours at 65° C. The DNA was recovered by phenol/chloroform extraction. Following primers were used in subsequent PCR reactions to amplify promoter regions of GATA4 and GATA6 containing the Ronin binding motif: (GATA4-f) 5′-GCA GAC CAG ATG CTG GAA GT-3′ (SEQ ID NO. 61) and (GATA4-r) 5′-TTT TCT CCG GTC CTG ATG TC-3′ (SEQ ID NO. 62); (GATA6-f) 5′-GCC ACA CAC ACA CCC TTG T-3′ (SEQ ID NO. 63) and (GATA6)-R 5′-AAG GCA AGG CAT CCT GAC TA-3′ (SEQ ID NO. 64). (Oct4-f) 5′-GGA TGG CAT ACT GTG GAC CT-3′ (SEQ ID NO. 65) and (Oct4-r) 5′-AGT TGC TTT CCA CTC GTG CT-3′ (SEQ ID NO. 66); (Nanog-f) 5′-CCA GTG GAG TAT CCC AGC AT-3′ (SEQ ID NO. 67) and (Nanog-r) 5′-GAA GTT ATG GAG CGG AGC AG-3′ (SEQ ID NO. 68).

### Stat3 Western Blot

For detection of STAT-3 and Phosho STAT-3, R1 and EF1α-Ronin cells (stably expressing Ronin) were plated at a density of 1.5×106 cells/10 cm dish in the presence of Lif, in the absence of Lif and in presence of 0.1 mg/ml aLif (BD Biosciences). Cells were lysed after 24 hours in the presence of PhosStop (Roche) phosphatase inhibitor. Equal amounts of protein were analyzed by Western blotting. The Stat3 antibody (BD Biosciences, #610189) was used in a 1:2500 dilution with the secondary anti-mouse-HRP antibody (Promega) diluted 1:2000, while Phospho-Stat3 antibody (Cell Signaling, #9131) was used in a 1:2000 dilution with the secondary anti-rabbit-1-IRP antibody (Abeam) diluted 1:2000.

### Doxycycline Induced Oct4 Knockdown During Transient Expression of Ronin

ZBHTc4.1 cells (Niwa, 2000, Nature Genetics 24:372-376) were plated at a density of 80000 cells per 10 cm dish coated with gelatin. Six hours after plating the cells were transfected with 24 mg of a 1:10 mixture of pCMV-GFP-N1 (Clontech) and pEF1a-loxP-Ronin-Ires-Neo or the pEF1-C control vector (Invitrogen) using Lipofectamine-2000 (Invitrogen) following the manufacturers instructions. 16 hours after transfection GFP positive cells were sorted and plated at 100000, 50000, 25000, 125000 and 6250 cells per 6 well. 20 hours after plating oct-4 knockdown was induced by treatment with 1 μg/ml doxycycline for 12 hours. Cells were washed twice with medium and then incubated for further 72 hours. LIF was included in the medium during the course of the experiment while no genetecin was added (the oct-4 transgene in ZBHTc4.1 is linked by an IRES to neomycin). Differentiation status was assessed after alkaline phosphatase staining as described.

### Establishment of ZBHTc4 (EF1α) and ZBHTc4.1 (EF1α-Ronin) Cell Lines

To establish a ZBHTC4.1 cell line stably expressing Ronin, a EF1α-Ronin-IRES-Puro containing vector was constructed and linear zed with SspI. ZBHTc4.1 cells were grown in the presence of 200 μg/ml genetecin and 2×107 cells were electroporated with 40 μg of linearized plasmid with a 950 μF and 220 V pulse. Cells were incubated on ice for 10 minutes and plated on three 10 cm dishes in medium without antibiotics. Puromycin selection was started 24 hours after plating at a concentration of 2.5 μg/ml Puromycin, which was reduced to 1 μg/ml after 4 days. Puromycin resistant colonies were picked after 11 days and expanded. The corresponding control vector pEF1a-IRES-PURO was used to generate the control cell line in parallel. 200 μg/ml genetecin were included in the medium to maintain oct4 expression.

### Doxycycline Induced Oct-4 Knockdown Under Stable Expression of Ronin

ZBHTc4 [EF1α] and ZBHTc4.1 [EF1α-Ronin] cells were plated at a density of 15.000 cells per 6 well. 24 h later, oct-4 knockdown was induced by addition of 1 μg/ml doxycycline for 8, 24 or 72 hours. LIF was included in the medium during the course of the experiment while genetecin was excluded (the oct-4 transgene in ZBHTc4.1 is linked by an IRES to neomycin). Differentiation status was assessed after alkaline phosphatase staining as described.

### Cell Proliferation Assay

For the cell proliferation assay 2000 cells per 6 well were plated and Ronin siRNA knockdown was performed as described. 18 hours after transfection Ronin expression was assessed by qPCR as described. Cell proliferation was measured 1, 2, 3 and 4 days after siRNA treatment with the CellTiter 96® AQueous One Solution Cell Proliferation Assay (MTS)(Promega) following the manufacturers protocol

### Bioinformatic Analysis

Ronin ChIP-derived reads were realigned to mm8 using an iterative version of ELAND to improve read count and all mapped reads (U.S. Pat. No. 4,059,673) were subjected to the standard Solexa error model. Bins were then evaluated with a Poissonian distribution, assuming that 50% of genomic bins were available for binding (conservative). Two cutoffs (p<10-7 and p<10-9), corresponding to 9 and 11 mapped reads, respectively, were used. The hierarchical clustering analysis of Ronin target genes by comparison with the targets for other factors was performed with the Cluster3.0 software. GSEA was conducted with GenePattern; the data set for differentiated ES cells (by EB formation) was obtained from Stembase (www.Stembase.ca). The DAVID tool was used to place genes in GO categories (http://david.abcc.ncifcrf.gov), while the PANTHER tool was implemented for PANTHER categories (http://www.pantherdb.org).

### Generation of Induced Pluripotent Stem Cells or Self-Renewing Cells

Reprogramming of MEFs to form iPS cells or iSCs followed the protocol of Takahashi et. al (Takahashi and Yamanaka, 2006, Cell 126:663-676). Briefly, Plat-E cells were transfected with different combinations of pMXs-Ronin, pMXs-Oct4, pMXs-c-Myc, pMXs-Klf4 and pMXs-Sox2 using the Fugene transfection reagent (Roche). Two days post transfection, the retroviral supernatants were filtered through 0.45 μm cellulose acetate filters. Primary MEFs (2×10⁵ per 10 cm2 plated on irradiated MEFs) were subsequently transfected with combinations of equal amounts of the filtered supernatants. The medium was changed every day and colonies were picked between 12 and 16 days post transfection.

Cell lines were established and subcloned. We genotyped the resultant cell lines after passage 3 and after subcloning using the following oligo combinations (also see Table S4): MAD459/MAD337 (pMXs-Oct4, ˜800 bp), MAD460/MAD337 (pMXs-c-Myc, ˜1600 bp), MAD460/MAD352 (pMXs-Klf4, ˜1400 bp), MAD460/MAD335 (pMXs-Sox2, ˜1200 bp), MAD459/MAD460 (pMXs-Ronin, ˜1000 bp) and MAD020/MAD223 (endogenous Ronin, 366 bp).

### MEF Isolation and Cre-Mediated Excision of RoninloxP/loxP

MEFs were isolated from RoninloxP/loxP animals and Ronin was removed by Cre-mediated recombination after transduction with adenovirus containing CMV-Cre-IRES-GFP (Ad-Cre) in the presence of Genejammer transfection reagent (Stratagene) as described earlier (Dejosez et al., 2008, Cell 133:1162-1174). Adenovirus containing CMV-GFP (Ad-GFP) served as a control. Two days after adenoviral transduction, cells were plated at a density of 2×105 cells per 10 cm2 for iPS cell induction as described above. Excision of loxP-flanked Ronin was verified by genotyping the cells with the oligos MAD357/MAD359 (Table S4), resulting in an approximately 900 bp PCR fragment if Ronin was excised successfully.

The results of the experiments presented in this Example are now described.

### Example 1

### Identification of Ronin by Yeast Two-Hybrid Screening

Previous studies showed that Nanog is targeted and cleaved by the pro-apoptotic enzyme Caspase-3 upon induction of ES cell differentiation (Fujita et al., 2008, in press), leading us to hypothesize that other, still unknown factors critical for ES cell pluripotency may be Caspase-3 targets as well. Yeast two-hybrid screening was performed on a human ES cell cDNA expression library, using constitutively active Caspase-3 (mCasp3rev) as bait. mCasp3rev spontaneously folds into its active conformation and recognizes and binds to target proteins, but no longer cleaves them owing to a C163S substitution. An estimated 32 million clones were screened, with 556 clones testing positive for interaction with the Caspase-3 mutant. Further study of a representative set of 286 clones, using rescued plasmids, digestion with restriction enzymes, and a validation assay, yielded 116 candidate genes. Subsequent sequencing and analysis with an in vitro transcription/translation Caspase-3 cleavage assay identified a cDNA whose protein product contained elements of a DNA-binding factor with striking similarities to the DNA-binding domain of the Drosophila P element transposase. This protein, termed Ronin for reasons given in the Introduction, proved to be an authentic target of Caspase-3 in further analyses (FIG. 8A).

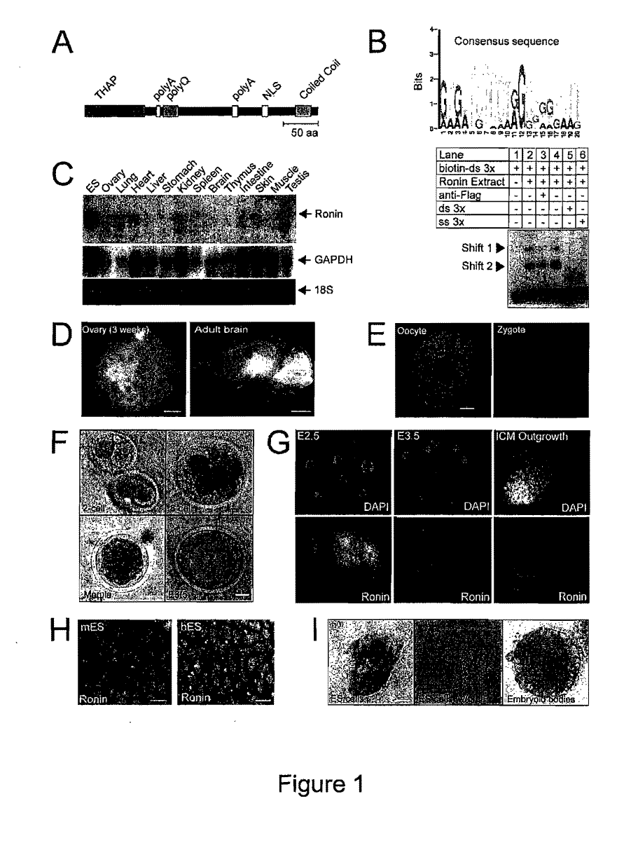

Characterization of the orthologous 305 residue Ronin protein encoded by the mouse cDNA (predicted length, 1809 bases) revealed a THAP domain at the N-terminus (FIG. 1A), which comprises a zinc-finger DNA-binding motif defined, in part, by a C₂CH signature (Cys-Xaa₂₋₄-Cys-Xaa₃₃₋₅₀-Cys-X-aa₂). There are also two polyalanine motifs, a polyglutamine tract (22 Qs), and a predicted coiled-coil structural domain at the C-terminus. A nuclear translocation signal (NLS) is located towards the C-terminus. A search for Ronin orthologs across multiple animal species showed exceptional conservation of the N- and C-termini, even among more distant species (e.g., humans vs. zebrafish, FIG. 8B, C). The most closely related nonvertebrate protein with a similar THAP domain is a transposase in the sea urchin (43% identity, seq XP_{—}790851.2), which is related to several Drosophila transposases, including the P element transposase and the MAP domain-containing protein THAP9 in humans and other primates.

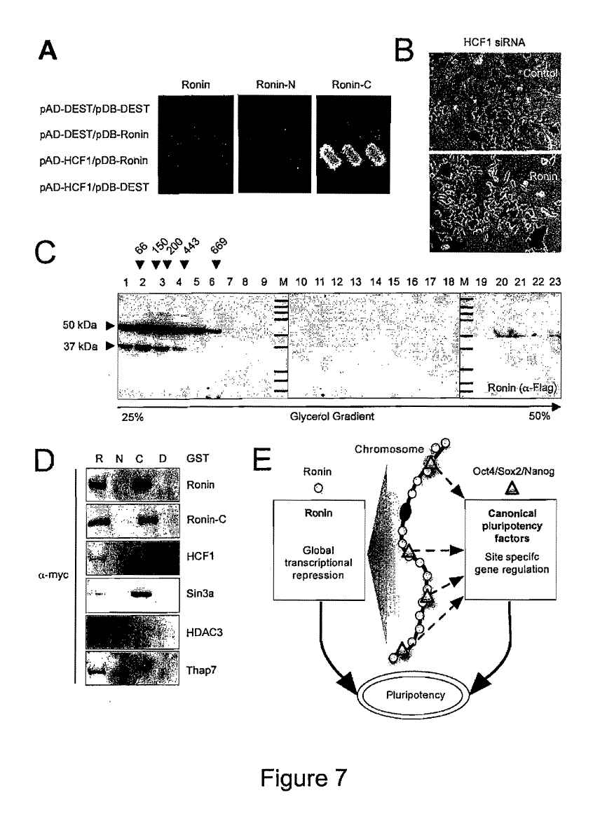

To determine the DNA sequence recognized by Ronin, the SELEX procedure was used with a mouse Ronin-His/V5 recombinant protein (see Experimental Procedures for details) to select random oligonucleotides for sequencing. A consensus sequence was identified (FIG. 1B, top panel) as well as a specific sequence that was represented three times in the sequenced pool, namely 3×. After the SELEX procedure, 104 sequences were analyzed, and a motif search identified a specific consensus (top) and a “3× sequence”. Binding of Ronin-Flag to the biotinylated 3× sequence was verified by gel-shift experiments that could be inhibited by the Flag antibody and either, double-stranded or single-stranded unlabeled competitor 3× molecules. Gel-mobility shift experiments confirmed that the 3× sequence is readily bound by Ronin (FIG. 1B, bottom panel). These data indicate that Ronin, like other proteins with a THAP domain, is a nuclear protein, as confirmed by immunofluorescence (FIG. 1G), and binds to DNA in a sequence-specific manner. The coiled-coil motif at the C-terminus may represent a second functional domain, as indicated by its capacity to bind directly to the HCF-1 protein (FIG. 7A, B).

### Example 2

### Ronin Expression Patterns

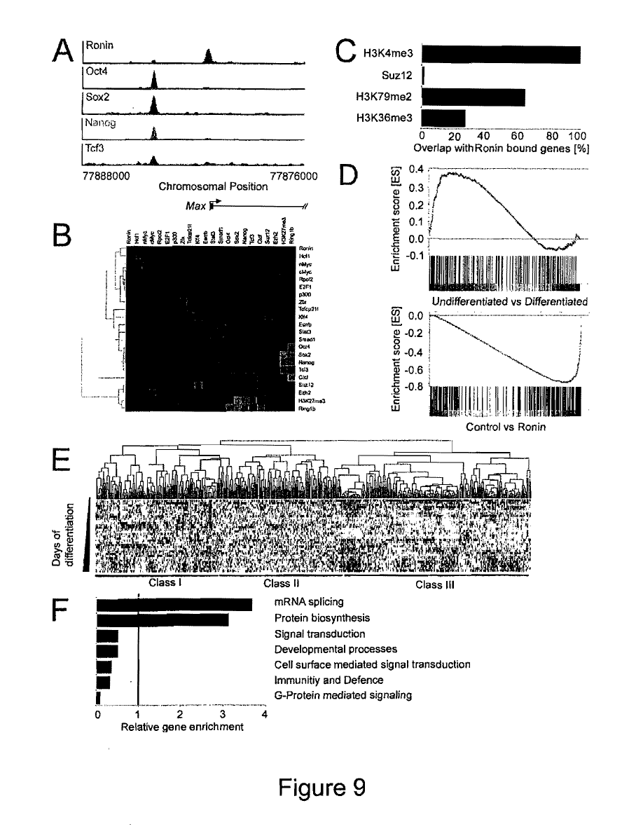

Northern blot analysis of multiple tissues in the mouse failed to detect appreciable expression of the Ronin gene, except in the mouse ES cell line used as a positive control (FIG. 1C). To further clarify the expression patterns of Ronin, a sensitive lacZ transgenic reporter mouse line in which a 3.3-kb genomic fragment representing the mouse Ronin promoter, was ligated to the open reading frame of the β-galactosidase gene. The resultant mouse line expressed β-galactosidase in tissues where the Ronin promoter was active, in a pattern similar to the expression of wild-type Ronin. As in the Northern blot analysis, Ronin was not abundantly expressed in adult tissues, with two exceptions: (i) ovary, which showed very strong positive staining in oocytes (FIG. 1D, left), and (ii) some areas of the brain, including hippocampus, olfactory bulb and Purkinje cells (FIG. 1D, right). To establish the subcellular compartment in which Ronin is found, an antibody was raised against Ronin (FIG. 9). Immunostaining with this antibody in adult ovaries showed localization of Ronin mainly in the ooplasm without any evidence of its presence in the nucleus (FIG. 1E, left). This pattern of staining contrasted with the detection of Ronin throughout the zygote (FIG. 1E, right). Using the lacZ animal model to assess Ronin expression during early embryonic development, we found that lacZ activity begins at the 2-cell stage, intensifies during the 8-cell and compact morula stages, but surprisingly, subsides in the blastocyst (FIG. 1F).

Immunostaining for Ronin protein was strongly positive in the nucleus of undifferentiated ES cells, and was distributed in an uneven pattern that primarily excluded DAPI-positive areas, suggesting that Ronin is an abundant nuclear protein associated with open chromatin (FIG. 1G). These results led us to study reporter gene activity in ES cells isolated from transgenic blastocysts to determine the temporal pattern of Ronin expression upon induction of differentiation. lacZ activity was detected in undifferentiated ES cells grown in the presence of a mouse embryonic fibroblast (MEF) feeder layer (FIG. 1H, left). When these cultures were transferred to gelatin-coated dishes and maintained in the absence of leukemia inhibitory factor (LIF) and MEFs or incubated as hanging drops to form embryoid bodies (EB), lacZ activity was virtually undetectable in the differentiated cells (FIG. 1H, middle and right). Overall, these findings indicate that Ronin expression is mainly restricted to pluripotent cells of the developing embryo and to oocytes and certain regions of the adult brain.

### Example 3

### Ronin Knockout Leads to Periimplantational Lethality

To test whether Ronin plays a critical role in early embryonic development, the gene in mouse ES cells was knocked out, generating Ronin^{+/-} mice (see Experimental Procedures). When Ronin^{+/-} male and female littermates were crossed, none of the 98 offspring at weaning age were Ronin^{-/-} demonstrating that a Ronin-null genotype is embryonically lethal. An estimated two-thirds of the offspring were Ronin^{+/-} (67 animals) and one-third were Ronin^{+/+} (31 animals), supporting a lethal phenotype. To determine the embryonic stage of lethality, we crossed Ronin animals and dissected and genotyped embryos at E7.5.

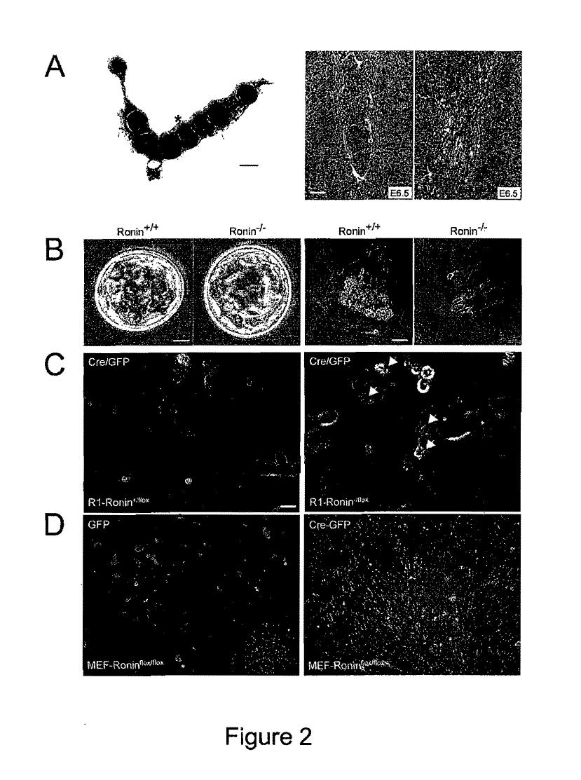

Of the +26 decidua examined, 7 (27%) were empty, 8 (31%) contained embryos that were Ronin^{+/+}, and 11 (42%) contained embryos that were Ronin^{+/-}. Empty swollen decidua were similar in size to those containing embryos, an indication that implantation and decidualization had proceeded normally (FIG. 2A, left). Crossing Ronin^{+/-} females with Ronin^{+/+} males did not yield empty decidua, and approximately half of the embryos were Ronin^{+/-} while the other half were Ronin^{+/+}, as expected. It is proposed that the Ronin^{-/-} embryos die either during or shortly after implantation, an outcome that was confirmed by the presence of residual embryonic tissue in empty decidua from uteri examined after crosses with Ronin^{+/-} mice (FIG. 2A, right).

Next, superovulated immature Ronin^{+/-} females were crossed with Ronin^{+/-} males, and 45 blastocyst-stage embryos were isolated. Genotyping of these blastocysts, identified nine (20%) as Ronin^{-/-}. Ronin^{+/-} females crossed with wild-type males produced the expected ratio of Ronin^{+/-} and Ronin^{+/+} blastocysts. Upon gross examination, the Ronin^{-/-} blastocysts were indistinguishable from Ronin^{+/+} and Ronin^{+/-} blastocysts (FIG. 2B, left). The inner cell masses (ICMs) of embryos resulting from additional Ronin^{+/-} and Ronin^{+/+} crosses showed outgrowth (90%) when cultured on gelatin-coated culture plates, in contrast to those from Ronin^{-/-} embryos, which either failed to proliferate or, in one instance, produced only a residual mass (FIG. 2B, right). It is proposed that Ronin is essential for maintenance and proliferation of the ICM.

### Example 3

### Ronin Knockout ES Cells Are Not Viable

The severe defects in ICM outgrowth in Ronin^{-/-} embryos implicated Ronin activity as a critical factor in both the derivation and propagation of ES cells. Hence, it was attempted to derive ES cells from crosses of Ronin^{+/-} mice. Although Ronin^{+/-} ES cell lines could be readily generated, it was not possible to obtain Ronin^{-/-} lines despite repeated attempts, indicating that Ronin activity is essential for generating ES cell lines in vitro. Even so, a knockout phenotype characterized by defects in the ICM would not necessarily militate against the growth and viability of cultured ES cells with conditionally deleted alleles. Thus, Ronin^{flox/flox} ES cells were generated, transfected with a Cre expression vector and sorted for Cre recombinase-positive cells (see Experimental Procedures). Among 110 genotyped subclones, the majority (90%) were Ronin^{flox/-}, with none lacking both alleles. Further testing of the Cre-transfected ES cells revealed a high rate of rapid apoptotic death (FIG. 2C), suggesting that Ronin knockout was lethal to ES cells under standard culture conditions. The Ronin^{+/flox} cells (green fluorescent) were viable while Ronin^{flox/-} cells died rapidly (arrow indicates typical morphology of apoptotic cells). Genotyping of 88 colonies failed to identify any Ronin^{-/-} cell. In contrast, when Ronin^{flox/flox} MEFs derived from E14.5-old embryos were isolated and treated with Cre adenovirus, nearly 100% of the cells were transduced, resulting in complete knockout of Ronin (FIG. 2D). Finally, the Ronin loxP allele was crossed into the Mx1-Cre background to generate Ronin^{flox/flox}-Mx1-Cre ES cell lines (Whyatt et al., 1993, Mol. Cell. Biol. 13:7971-7976) but the induction of Mx1-Cre did not lead to deletion of the Ronin allele in any experiment. After transfection with GFP adenovirus, all MEFs showed green fluorescence indicating successful transfection (left panel, insert shows phase contrast image of transduced MEFs). PCR-based genotyping of Cre-GFP-transfected cells confirmed successful Cre-mediated excision of both Ronin alleles, resulting in viable Ronin^{-/-} MEF cells. Together, these findings demonstrate a stringent requirement for Ronin in maintenance of the self-renewal property of ES cells, as well as in generation of the ICM during early embryogenesis.

### Example 4

### Forced Expression of Ronin Inhibits Differentiation

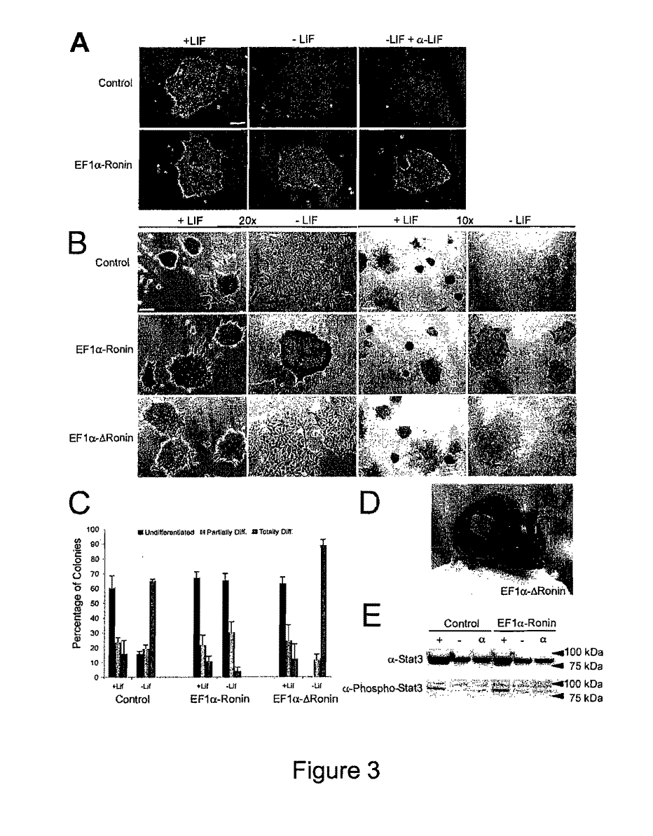

Because Ronin possesses several of the critical features of a pluripotency factor, we asked if its ectopic expression in ES cells would render them independent of LIF for self-renewal. In these experiments, stable ES cell lines were established expressing loxP-flanked Ronin under the influence of a constitutive promoter, EF1α. Western blot analyses were used to select several clones that expressed Ronin in ES cells and had normal morphology. To test the effects of Ronin overexpression on ES cell self-renewal, control ES cells and those ectopically expressing Ronin (maintained without LIF or with a LIF-blocking antibody) were plated at clonal densities and analyzed colony formation 4 days later. The vast majority of colonies overexpressing Ronin appeared morphologically unaffected by LIF removal or LIF inhibition, in contrast to ES cell controls, which were fully differentiated (FIG. 3A). To quantify this result, alkaline phosphatase staining was performed 4 days after clonal plating and determined the percentages of undifferentiated, partially differentiated and fully differentiated ES cell colonies. As expected, in the absence of LIF most of the control ES cell colonies were either partially (24%) or entirely (60%) differentiated, whereas two-thirds (65%) of the EF1α-Ronin ES cell colonies remained undifferentiated under the same conditions (FIG. 3B, C). Furthermore, there was essentially no background differentiation in EF1α-Ronin ES cell cultures. This remarkable example of LIF-independent maintenance of pluripotency was further evaluated at the functional level by culturing ES cells without LIF for 8 days and subsequently removing the Ronin transgene with Cre recombinase. All control ES cells differentiated relatively quickly, to the extent that no cells with typical ES cell morphology remained in the culture when they were split after 4 days. In sharp contrast, the Ronin-expressing ES cells formed abundant colonies and could be split after 4 days of culture without LIF. After a total of eight days in the absence of LIF, clones were expanded and the ectopic Ronin allele was removed by Cre transfection of expanded clones (EF1α-ΔRonin), these cells displayed properties indistinguishable from those of wild-type ES cells, including monolayer differentiation in medium without LIF (FIG. 3B, C) and the ability to generate chimeric animals upon injection into blastocysts, similar to control cells (FIG. 3D). These results indicate that the absolute differentiation block was not due to a secondary mutation in EF1α-Ronin ES cells. They also suggest that the pluripotency sustained by ectopic expression of Ronin is reversible.

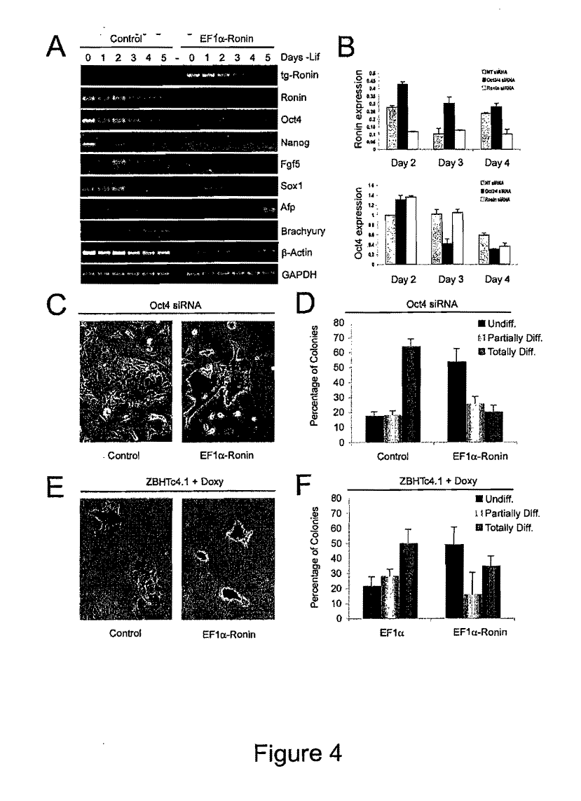

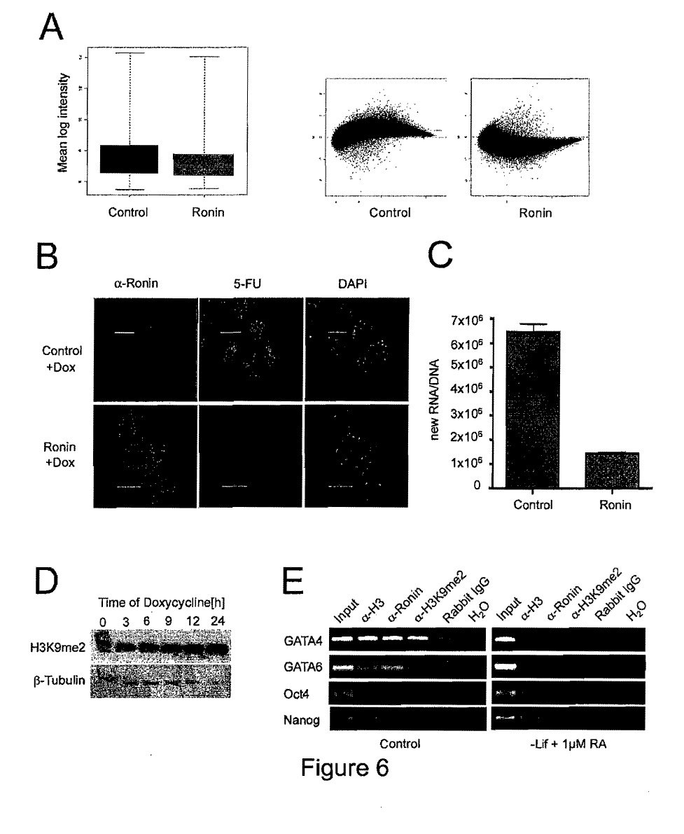

To assess the effects of constitutive expression of Ronin on (i) known pluripotency factors, (ii) marker genes for all three germ layers, (iii) and extraembryonic tissues, RNA was isolated from ES cells on days 1 through 5, after they were plated at low densities in medium without LIF. RT-PCR analysis revealed two provocative but conflicting results (FIG. 4A): (i) virtually all differentiation markers were inhibited upon withdrawal of LIF, indicating that forced expression of Ronin inhibits differentiation, similar to findings with the teratocarcinoma formation assays (see below), whereas (ii) Oct4 especially and to some extent Nanog were simultaneously down regulated faster than in control cell lines. Interestingly, even the amount of RNA for the housekeeping gene, β-actin, was significantly reduced in repeated experiments. This finding suggests that the repressive function of Ronin is not limited to specific developmental genes but extends widely over the transcriptome, a prediction tested in FIG. 6.

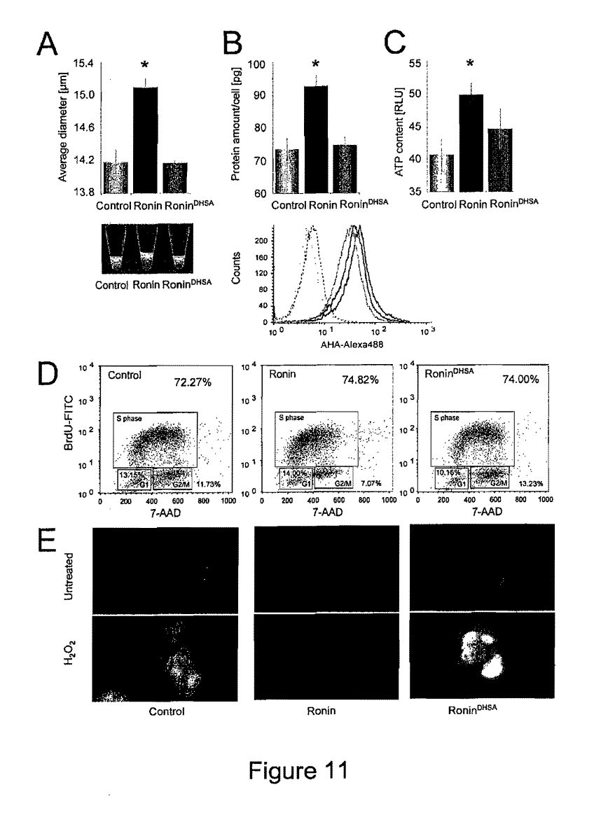

To determine if knock-down of Oct4 affects the expression of Ronin, siRNA experiments were performed in which Oct4 was rapidly down regulated while Ronin was not affected (FIG. 4B). These results suggest that Ronin acts independently of Oct4 and Nanog to maintain pluripotency, an interpretation supported by the findings of Ivanova et al. (FIG. 11; Ivanova et al., 2006, Nature 442:533-538). Functional proof that Ronin expressing cells can self-renew independently of Oct4 expression came from experiments in which we transfected control and Ronin-expressing cells with siRNA against Oct4. In contrast to controls, the reduction of Oct4 expression had no effects on cell morphology or differentiation (FIG. 4C). The results of constitutive expression of Ronin indicate that this factor maintains pluripotency independently of the LIF/Stat3 pathway and the Oct4/Sox2/Nanog axis.

### Example 5

### Ectopic Expression of Ronin is Tumorigenic

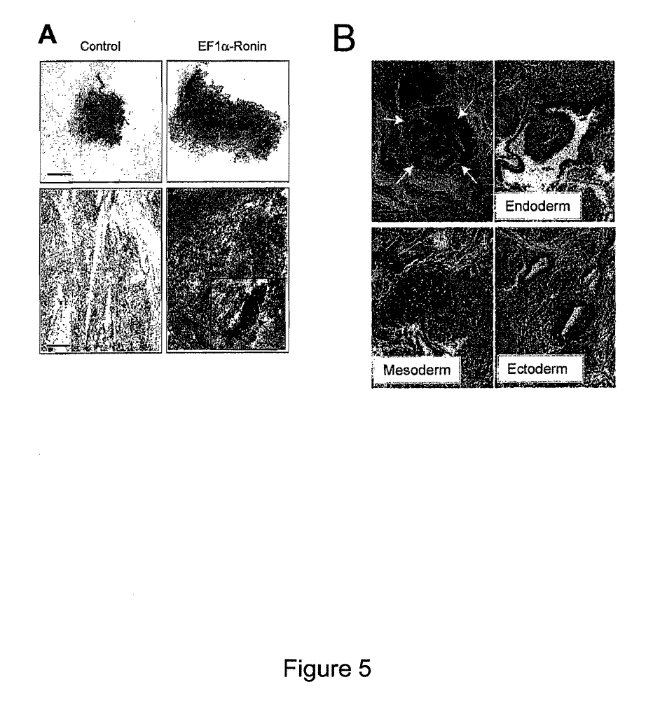

If Ronin truly acts as an antidifferentiation factor in ES cells, its overexpression should be associated with strong tumorigenicity. Thus, to assess teratocarcinoma formation, control ES cells and EF1α-Ronin ES cell lines were injected into the hind-leg quadriceps muscle of SCID immunocompromised mice. Animals injected with control ES cells displayed teratocarcinomas of the expected size by 17 days, while those injected with EF1α-Ronin had substantially larger tumors (2.8 cm versus 1.8 cm) (FIG. 5A, top right), suggesting that increased Ronin activity triggers expansion of the stem cell pool, leading to more robust teratocarcinoma formation prior to differentiation. Histologic examination of the teratocarcinomas derived from both control ES and Ronin-expressing EF1α-Ronin ES cells revealed differentiation into all three germ layers in both contexts (FIG. 5B), However, a substantial number of undifferentiated cell clusters in the EF1α-Ronin tumors were noticed that resembled embryonic carcinoma cells (FIG. 5B, top left). This impression was supported by immunostaining results indicating Oct4-positive cell clusters among the EF1α-Ronin ES cells but not the control ES cell line (FIG. 5A, bottom panels). Thus, the enhanced tumorigenicity of ES cells constitutively expressing Ronin appears to stem from the antidifferentiation effects of this factor, supporting its candidacy as a major regulator of the pluripotent state.

### Example 6

### Ronin is a Transcriptional Regulator that Acts Through a Multimeric Protein Protein Complex Containing HCF-1

How does Ronin maintain the pluripotency of ES cells? The most likely mechanism, based on Ronin's antidifferentiation effects and the epigenetic silencing activity of other THAP domain proteins (Roussigne et al., 2003, Trends Biochem. Sci. 28:66-69; Mcfarlan et al., 2005, J. Biol. Chem. 280:7346-7358), is transcriptional regulation of multiple genes that are either directly or indirectly involved in differentiation. To test this hypothesis, gene expression profiling of control ES cells versus ES cells transiently transfected with a Ronin-overexpressing construct was performed. This comparison (FIG. 6A) showed a striking regulation of the transcriptome of Ronin-transfected cells. A Ronin inducible cell line was generated by inserted the Ronin-encoding cDNA, under the control of a tetracycline-inducible promoter, upstream of the Hprt locus in A172loxP ES cells and compared the kinetics of RNA transcription in control versus Ronin-expressing cells stained with an anti-bromodeoxyuridine antibody against 5-fluorouracil (5-FU). There was a clear and rapid loss of newly synthesized RNA in cells that overexpressed Ronin (FIG. 6B). This outcome was confirmed by the results of a ³H-uridine pulse-chase incorporation assay (FIG. 6C). Finally, Western blot analysis to detect histone H3 dimethylation at lysine 9 (H3K9me2) in our Ronin-inducible cell line showed a large and rapid increase in the methylation of this protein over time (FIG. 6D).

To pursue the idea that Ronin exerts its antidifferentiation effects through regulation of gene expression, an immunoprecipitation strategy was devised to identify protein complexes associated with FLAG-tagged Ronin in ES cells. Putative interaction partners were separated by SDS gel electrophoresis and the protein bands subjected to protein tandem mass spectometry analysis. Of 80 candidate proteins, 32 were selected for further evaluation by a directional yeast-two hybrid system. In this approach, full-length Ronin as well as two truncated forms carrying the N-terminus (Ronin-N) or the C-terminus (Ronin-C) were tested for their ability to bind directly to selected putative interaction partners (FIG. 7A). The only direct interaction that was identified was between Ronin-C and host cell factor 1 (HCF-1). Retrospective analysis of the Ronin sequence revealed a previously described HCF-1 interaction motif (Freiman and Herr, 1997, Genes Dev. 11:3122-32127) at the C-terminus of the molecule, making this protein a likely direct target of Ronin. Wysocka et al. (2003, Genes Dev. 17:896-911) isolated a multimeric HCF-1-containing protein complex from HeLa cells by taking advantage of the glycoprotein properties of HCF-1; a similar strategy was applied to purify a Ronin protein complex from EF1α-Ronin ES cells. Comparison with the elution peaks of protein standards of known sizes, detected by Coomassie blue staining suggested that Ronin functions within a very large (>2 MDa) protein complex (FIG. 7B). To identify some of the components of this complex, the same set of proteins used for the yeast-two hybrid evaluation were selected and modified for use in co-transformation assays. Thus, 293 cells were co-transfected with GST-tagged variants of Ronin, Ronin-C or Ronin-N and with Myc-tagged variants of the putative interaction partners. With this strategy, the binding of Ronin to HCF-1 via the C-terminus was confirmed. Other confirmed protein interaction partners (FIG. 7C) were Ronin itself (via homodimerization through the C-terminus); THAP7, another THAP domain protein (via heterodimerization through the N-terminus); and Sin3A (C-terminus) and HDAC3 (N-terminus). Thus, Ronin acts through a large HCF-1-containing protein complex that can modulate gene expression over broad regions of the transcriptome.

### Example 7

### Ronin Binds to a Highly Conserved Promoter Element

To understand how Ronin exerts its regulatory activities in ES cells its DNA-binding sites were identified in self-renewing mouse ES cells using the ChIP-seq method (chromatin immunoprecipitation coupled with Illumina sequencing). The mapping results in self-renewing mouse ES cells (FIG. 8A) revealed 346 Ronin-bound DNA sequences, most of which were located at or immediately upstream of the transcriptional start sites of genes (FIG. 8B). Alignment analysis identified a Ronin-binding motif (YYACAWRTCCCT; SEQ ID NO. 69) in 260 of the target genes (FIG. 8C). This functional element was highly enriched compared with random sequences (p<10-909, FIG. 1C), and partly resembled the Ronin binding sequence (‘3x’) that had been previously determined by the SELEX method (Dejosez et al., 2008, Cell 133:1162-1174). Intriguingly, the Ronin target sequence closely matched an orphan promoter sequence, designated M4 (ACTAYRNNNCCCR; SEQ ID NO. 70; FIG. 1C), whose conservation rate in humans (61%). The M4 sequence is notable for another reason: in contrast to most highly conserved regulatory units in the human genome, it lacked a recognized binding factor before this analysis. Gel-shift (EMSA) experiments (FIG. 8D) and ChIP with a Ronin specific antibody, followed by PCR analysis of selected target gene regions (FIG. 8E), confirmed the sites found to be specifically targeted by Ronin or a Ronin-containing protein complex.

Because Ronin had appeared to interact directly with the Hcf-1 protein in earlier experiments (Dejosez et al., 2008, Cell 133:1162-1174), ChIP was also performed with an antibody raised against Hcf-1 followed by PCR analysis for the same genes targeted by Ronin. The results (FIG. 8E) showed a clear overlap between positive signals obtained by Ronin and Hcf-1 precipitations, indicating that Ronin indeed recruits Hcf-1 directly to target genes. Thus, the data show that Ronin functions as an authentic DNA-binding transcription factor, whose target sequence is a highly conserved promoter element with likely biological significance in mammalian cells.

### Example 8

### Ronin Activates Gene Expression with Only Minimal Contributions from Oct4, Sox2 and Nanog

The preference of Ronin for the novel binding site described above suggests that it might exert some or all of its function independently of Oct4 and other canonical pluripotency factors. To test this prediction, all Ronin binding sites were tested for the presence of Oct4, Sox2, Nanog and Tcf3. Of these 346 target gene promoters, 92 were bound by at least two of these transcription factors, while 174 were bound by at least one factor (typically, when any factor was present at significant levels, the others were also present but below the p<10⁻⁹ threshold). Despite this co-occupancy pattern, the binding sites recognized by Oct4, Sox2, Nanog and Tcf3 were consistently distinct from those occupied by Ronin, as illustrated by the combinatorial binding pattern of Ronin, Oct4, Sox2, Nanog and Tcf3 to the Max oncogene promoter (FIG. 9A). Indeed, hierarchical clustering analysis of promoter occupancies by 20 factors associated with pluripotency and Ronin/Hcf1 revealed clustering primarily with Zfx, p300, E2f1 and c-Myc rather than Oct4, Sox2 and Nanog (FIG. 9B). Further analysis of the Ronin-occupied promoter regions (FIG. 9C) showed that Ronin is closely associated with promoters containing histone H3K4me3-modified nucleosomes, a mark of genes that experience transcription initiation (100% overlap), and with H3K36me3 (28%) and H3K79me2 (80%), both marks of genes that are fully transcribed. In contrast, there was essentially no overlap with Suz12 (0.02%), a component of the Polycomb Repressive Complex 2, which catalyzes the H3K27me3 mark associated with transcriptionally repressed bivalent domains (Bernstein et al., 2006, Cell 125:315-326), suggesting a very strong negative correlation (binomial p<10-9).

To confirm that Ronin positively regulates the transcription of its target genes, a gene set enrichment analysis (GSEA) of Ronin target genes in ES cells was done and compared with cells from embryoid bodies (FIG. 9D, top panel), which indicated that Ronin-controlled genes were generally highly expressed and significantly overrepresented.

The Ronin transcriptional program in wild-type ES cells was compared with that in cells ectopically and stably overexpressing Ronin (EF1α-Ronin ES cells). Again, GSEA showed an overrepresentation of Ronin target genes in EF1α-Ronin ES cells, suggesting that Ronin indeed positively affects the expression of many target genes (FIG. 9D, bottom panel). This interpretation implies that the acute upregulation of Ronin under otherwise steady-state condition provokes a dominant-negative effect of Ronin function, similar to published observations on other Thap domain containing proteins (Cayrol et al., 2007, Blood 109:584-594). Further analysis of the 346 genes controlled by Ronin revealed that 213 (62%) were highly expressed in ES cells, undergoing rapid downregulation upon differentiation (FIG. 9E). Together, these results indicate that Ronin binding is closely linked to gene activation. Whether this function is influenced by Oct4, Sox2, Nanog and Tcf3 co-occupancy of Ronin target gene promoters cannot be addressed with the data presented here. However, the close proximity of the Ronin binding sequence to the transcriptional start sites of Ronin-regulated genes, and the lack of overlap between Ronin targets identified in this study and those downregulated upon Oct4 knockdown [only 7 of 1065 Oct4-regulated genes are direct Ronin targets (Ivanova et al., 2006. nature 442:533-538)] suggest minimal interaction between these canonical pluripotency factors and Ronin during the self-renewal of ES cells.

### Example 9

### Ronin Regulates a Genetic Program Associated with ES Cell Perpetuation

The striking affinity of Ronin for the M4 regulatory motif suggests that Ronin is likely to control a subset of biologically important genes. Thus, using the PANTHER biological process classification tool, the functional categories of both Ronin and Oct4/Sox2/Nanog target genes was determined. Protein biosynthesis was the most significantly overrepresented category in the Ronin target gene group as compared with the Oct4/Sox2/Nanog group (p<3.12×10-2), whereas developmental process and signal transduction were the most significantly underrepresented (p<1.78×10-5 and p<4.37×10-4 respectively; FIG. 9F). Closer examination of these Ronin targets using specific gene ontology (GO) subcategories yielded a more informative functional portrait. Ribonucleoprotein complex components (p=8.24×10-5) and mitochondrial part (p=1.8×10-2), representing genes with known functions in cell growth, were significantly enriched.

Within these subcategories, Ronin bound to ribosomal protein-encoding genes, such as Rpl36, Rpl18 and RplS3 (manual inspection revealed that as many as 30% of the ribosomal protein-encoding genes were occupied by Ronin), and strikingly two key subunits of RNA polymerase I (Rpol-2 and Rpol-4). These results are important because alterations in ribosomal biosynthesis can have profound effects on the metabolome of any cell, including stem cells (Moss and Stefanovsky, 2002, Cell 109:545-548; Tsai and McKay, 2002, Genes Dev. 16:2991-3003). Ronin also bound specifically to a subset of mitochondrial rRNAs (Mrp125, 32, 34 and 50), mitochondrial translation factors (Tufm) and rate-limiting members of the oxidative phosphorylation cascade (Atp5j, NADH dehydrogenase, Cox7c, etc.), suggesting its involvement in the control of energy production in ES cells. Finally, sustained self-renewal of ES cells would be expected to require efficient DNA repair and protection of telomeres to avoid untimely cell death. Thus, it was not surprising to find that the genes activated by Ronin included a substantial number of genes encoding some of the most conserved proteins involved in DNA maintenance and repair (e.g., Rad51, p53, SirT2 and Brca1), which are thought to have a role in stem cell function (Liu et al., 2009, Genomics, Epub ahead of print). Ronin also targeted four genes (Trf2, Wm, Chk2 and Ercc1) that specify the Trf2 telomere complex, which lies at the heart of stem cell immortality and function (de Lange, 2002, Oncogene 21:532-540).

Given its apparent essential role in ES cell pluripotency, Ronin would likely need to coordinate its activities with other transcription factors and chromatin regulators. Accordingly, Ronin binds to its own promoter as well as to the promoter of the pluripotency factor Sal14, which is able to positively regulate canonical pluripotency factors (Wu et al., 2006, J. BIol. Chem. 281:24090-24094); the promoter encoding the B1 element of the NF-κB complex; Gcnf, a known silencer of pluripotency factors (Gu et al., 2005, Mol. Cell. Biol. 25:8507-8519); Hcfc1R, the regulator of Hcf-1 (Mahajan et al., 2002, J. Biol. Chem. 277:44292-44299); and Max, which encodes the binding partner of c-Myc (Prendergast et al., 1991, Cell 65:395-407). These findings suggest that Ronin relies on autoregulatory, feed-forward and feed-backward loops, and may engage in cross-talk with other pluripotency factors. It appears important that Ronin did not bind to any classical cell cycle genes, indicating that it may exert its control of pluripotency without affecting cell cycle progression. Taken together, the results in this section indicate that Ronin controls the transcription of a relatively small number of genes with specific functions related to protein metabolic activity, mitochondrial activity and DNA/telomere maintenance but not to differentiation or cell division.

### Example 10

### Hcf-1 Modifies Ronin Function

To gain insight into the full extent of overlap between binding sites occupied by both Ronin and Hcf-1, ChIP-seq was performed with an Hcf-1 antibody (Wilson et al., 1993, Cell 74:115-125). After background subtraction and binding peak analysis, 127 target promoters were identified with high confidence that were co-occupied by Hcf-1 and Ronin (FIG. 10A). Even when the Hcf-1 signal did not attain significance by these conservative cutoff criterion, it was still possible to detect a substantial binding peak, suggesting that nearly all promoters recognized by Ronin are co-occupied by Hcf-1. This finding supports the working hypothesis that the transcriptional regulatory activity of Ronin is mediated through a binding complex that includes Hcf-1 and chromatin-modifying proteins (Dejosez et al., 2008, Cell 133:1162-1174).

Ronin and Hcf-1 interact at a common regulatory motif in promoters, but exactly how this interaction affects the transcriptional activity of Ronin is uncertain. Ronin contains a DHSY copy (Dejosez et al., 2008, Cell 133:1162-1174) of the previously defined HCF-1 binding motif D/EHxY (Freiman and Herr, 1997, Genes Dev. 11:3122-3127; Lu et al., 1998, J. Virol. 72:6291-6297) and mutated a conserved residue essential for binding: 246Y->246A, resulting in RoninDHSA (FIG. 10B, top panel). By yeast two-hybrid assay, this change completely abolished the ability of Ronin to interact with Hcf-1 (FIG. 10C). To interrogate how the inability of Ronin to recruit Hcf-1 might affect the transcriptional activity of Ronin in ES cells, stably transfected ES cell lines overexpressing either wild-type Ronin (EF1α-Ronin ES cells) or the mutant form, which is not capable of binding Hcf-1 (EF1α-RoninDHSA ES cells were generated). Because expression of Ronin effectively supports the self-renewal of ES cells in leukemia inhibitory factor (Lif)-free medium (Dejosez et al., 2008, Cell 133:1162-1174), the effect of Hcf-1 on this property was tested by plating control ES cells, EF1α-Ronin ES cells and EF1α-RoninDHSA ES cells at clonal densities in medium with or without LIF (FIGS. 10D and 10E). As previously reported, Ronin robustly made Lif nonessential for ES cells. However, EF1α-RoninDHSA ES cells still differentiated, suggesting a requirement for Ronin to interact with Hcf-1 to exhibit its antidifferentiation effect (FIGS. 10D and 10E).

Thus, after binding to Ronin at regulatory sites on target gene promoters, Hcf-1 appears to amplify Ronin function.

### Example 11

### Ronin Controls Cell Growth and DNA Protection Properties

To validate the apparent major role of Ronin in meeting the metabolic requirement of proliferating ES cells, we focused our attention on cell size, reasoning that this parameter would be highly sensitive to fluctuations in Ronin activity. In experiments to determine the diameter of EF1α-Ronin ES cells and EF1α-RoninDHSA ES cells compared with wild-type controls, we found that EF1α-Ronin ES cells were significantly enlarged (>6.4% increase in size, p=5.63×10-4; FIG. 4A, top panel), a change that was clearly visible in cell pellets adjusted for cell number (FIG. 4A, bottom panel). Importantly, ES cells expressing EF1α-RoninDHSA did not show an increase in cell size, confirming the requirement for Hcf-1 in optimal Ronin function. EF1α-Ronin ES cells also had a different shape, appearing rounder with a more prominent nucleolus (Figure S3D). We also measured the amount of protein per cell (FIG. 4B, top panel) as well as protein translation capacity per cell (FIG. 4B, bottom panel) demonstrating that Ronin-overexpressing cells have significantly more protein content and translational activity per cell than do RoninDSHA and wild-type ES cells (p=1.64×10-2). Finally, we found that the amount of ATP per cell was significantly higher in EF1α-Ronin ES cells as compared with wild-type ES cells (p=3.92×10⁻³) or EF1α-RoninDHSA ES cells (FIG. 4C). This increase in cell size/protein metabolism was not due to a difference in cell cycle kinetics, as similar numbers of control ES cells and EF1α-Ronin ES cells were in S phase (FIG. 4D). We also interrogated the DNA protection capacity of wildtype, EF1α-Ronin and EF1α-RoninDHSA ES cells (FIG. 4E) by staining against p-H2AX. Even before treatment, the EF1α-Ronin ES cells contained a smaller number of p-H2AX foci than did control cells, and this difference became more pronounced after the cells were challenged with H2O2 to introduce DNA double-strand breaks.

### Example 12

### Ronin Restores a Self-Renewal Program Without Activating the Canonical Pluripotency Network

Ectopic expression of Oct4, Sox2, Myc and Klf4 has proved to be a robust strategy of reprogramming somatic cells to a pluripotent state (Takahashi et al., 2007, Cell 131:861-872; Takahashi and Yamanaka, 2006, Cell 126:663-676; Wernig et al., 2007, Nature 448:318-324). The mechanism by which iPS cells arise after these transcription factors are introduced remains unclear, but presumably involves the restoration of a transcriptional network that will ensure the negative regulation of differentiation (Mikkelsen et al., 2008, Nature 454:49-55). Thus, if Ronin acts principally on genes essential for the perpetuity of ES cells, as our data argue, its substitution for one or more canonical pluripotency factors in a standard iPS cell induction protocol should impose the self-renewal state of pluripotent cells generated from fibroblasts without affecting their developmental stage. To test this prediction, we first established the role of Ronin during reprogramming with canonical pluripotency factors. We observed a low level of Ronin expression in mouse embryonic fibroblasts (MEFs) and a sharp increase in Ronin mRNA 4 days after virus transduction (FIG. 5A). This finding was accompanied by the detection of low amounts of Ronin protein in both the cytoplasm and nucleus in regular MEFs and on day 3 by a striking increase in the amount localized in the nucleus (FIG. 5A), consistent with previous observations (Dejosez et al., 2008, Cell 133:1162-1174). We then generated Ronin−/− MEFs and subjected them to the classical four-factor reprogramming combination. After only a few days post-transduction, Ronin−/− MEFs formed a significantly lower number of colonies than did cells with an intact Ronin gene (FIG. 5B, left panel). Examination of 40 of these colonies revealed that none retained a homozygous Ronin deletion, in contrast to the initial population of MEFs, in which most of the cells had the homozygous knockout (FIG. 5B, right panel). This result indicates that Ronin is essential for the generation of iPS cells with classical pluripotency factors.

In experiments to test whether Ronin can induce self-renewal in MEFs without activating the Oct4/Sox2/c-Myc/Klf4 transcriptional network, we found that Ronin together with either Sox2/c-Myc/Klf4 or c-Myc/Klf4 can give rise to alkaline phosphatase-positive colonies (FIG. 5C) whereas Sox2/c-Myc/Klf4 or c-Myc/Klf4 without Ronin failed to propagate. Cell lines established in the above manner displayed the proliferative kinetics and colony morphology typically associated with primary iPS or ES cell lines, before and after splitting (FIGS. 5D and S4A). Nonetheless, these induced self-renewing clones, designated iSCRSMK or iSCRMK to specify their origin, did not express any of the typical pluripotency-associated genes (FIG. 5E). Further analysis of these cells, using standard embryoid body differentiation protocols, revealed that they did not differentiate in the manner of iPS or ES cells. Interestingly, after injection into immunocompromised mice, the iSCs formed rapidly growing tumors, which by histological analysis consisted of undifferentiated, rapidly growing cells that had lost all features of fibroblasts (FIG. 5F). Genotyping demonstrated integration of the Ronin retroviral vector in all iSC lines (Figure S4A), suggesting that Ronin plays a critical role in the initiation of self-renewal. To determine the extent to which Ronin overexpression reactivates a self-renewal program in fibroblasts, we performed microarray analysis that showed overexpression of a significant number of genes (294) in iSCs versus MEFs and ES cells (FIGS. 5G and 5H).

Gene expression profiling and subsequent principle component analysis of iSC lines, MEFs and ES cells showed that all iSCs had similar expression profiles that differed from the MEF and ES cell profile (Figure S4B). These results provide compelling evidence for a Ronin-controlled genetic program in ES cells that sustains self-renewal and is distinct from the transcriptional network regulated by canonical pluripotency factors.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of maintaining the self-renewal capacity of an embryonic stem (ES) cell, said method comprising contacting said cell with an effective amount of Ronin or a Ronin activator, wherein when said Ronin or Ronin activator contacts said ES cell, said Ronin or Ronin activator maintain the self-renewal capacity of said ES cell.

2. The method of claim 1, wherein said Ronin or Ronin activator is provided to said ES cell exogenously.

3. The method of claim 2, wherein said activator is selected from the group consisting of a protein, a nucleic acid, and a small molecule.

4. The method of claim 1, wherein said Ronin or said Ronin activator is expressed by said ES cell.

5. The method of claim 4, wherein expression of said Ronin activator is controlled by a promoter.

6. The method of claim 5, wherein said promoter is an inducible promoter.

7. The method of claim 1, wherein said ES cell is a mammalian embryonic stem cell.

8. The method of claim 7, wherein said ES cell is a human embryonic stem cell.

9. A method of maintaining self-renewal capacity of an ES cell, said method comprising regulating expression of Ronin in said cell by an inducible promoter, wherein when said Ronin expression is increased, said ES cell does not differentiate.

10. The method of claim 9, wherein said ES cell is a mammalian embryonic stem cell.

11. The method of claim 10, wherein said ES cell is a human embryonic stem cell.

12. A method of reversibly preventing the differentiation of an ES cell, said method comprising the steps of:
a) conditionally expressing Ronin or a Ronin activator in said ES cell, wherein said conditional expression of said Ronin or Ronin activator is under the control of an inducible promoter;
b) contacting said ES cell with an inducing agent, wherein when said inducing agent contacts said cell, said inducing agent activates said inducible promoter, thereby increasing expression of said Ronin or Ronin activator in said ES cell, wherein said ES cell does not differentiate;
c) and, optionally, removing said inducing agent, whereby said Ronin or Ronin activator expression is reduced in said ES cell, and said ES cell differentiates.

13. The method of claim 12, wherein said expression of Ronin is controlled by an inducible promoter.

14. The method of claim 12, wherein said cell is a mammalian embryonic stem cell.

15. The method of claim 14, wherein said cell is a human embryonic stem cell.

16. A composition comprising an ES cell having an inducible promoter, wherein said promoter regulates the expression of Ronin or a Ronin activator by said ES cell.

17. The method of claim 16, wherein said activator is selected from the group consisting of a protein, a nucleic acid, and a small molecule.

18. The method of claim 16, wherein said cell is a mammalian embryonic stem cell.

19. The method of claim 16, wherein said cell is a human embryonic stem cell.
